(19) United States
(12) Patent Application Publication
(10) Pub. No.: US 2016/0009752 A1
(43) Pub. Date: Jan. 14, 2016

(54) PREGNANE-OXIMINO-AMINOALKYLETHERS AND PROCESS FOR PREPARATION THEREOF, USEFUL AS ANTIDIABETIC AND ANTIDYSLIPIDEMIC AGENTS

(71) Applicant: COUNCIL OF SCIENTIFIC & INDUSTRIAL RESEARCH, Rafi Marg, New Delhi (IN)

(72) Inventors: Prem Chandra VERMA, Lucknow (IN); Jyoti GUPTA, Lucknow (IN); Dharmendra Pratap SINGH, Lucknow (IN); Varsha GUPTA, Lucknow (IN); Hari Narayan KUSHWAHA, Lucknow (IN); Anamika MISRA, Lucknow (IN); Neha RAHUJA, Lucknow (IN); Rohit SRIVASTAVA, Lucknow (IN); Natasha JAISWAL, Lucknow (IN); Ashok Kumar KHANNA, Lucknow (IN); Akhilesh Kumar TAMRAKAR, Lucknow (IN); Shio Kumar SINGH, Lucknow (IN); Anil Kumar DWIVEDI, Lucknow (IN); Arvind Kumar SRIVASTAVA, Lucknow (IN); Ram PRATAP, Lucknow (IN)

(21) Appl. No.: 14/763,480

(22) PCT Filed: Jan. 24, 2014
(86) PCT No.: PCT/IN2014/000055

§ 371 Date: Jul. 24, 2015

(30) Foreign Application Priority Data
Jan. 24, 2013 (IN) O193/DEL/2013

(51) Int. Cl. C07J 41/00; C07J 43/00; A61K 31/57

(52) U.S. Cl. CPC C07J 41/005; A61K 31/57; C07J 43/003

(57) ABSTRACT

The present invention relates to the synthesis of pregnane-oximino-aminoalkyl-ethers and their antidiabetic and antidyslipidemic activities. More particularly, the invention relates to the synthesis of compounds of formula 3 and biological profile thereof. Further the invention relates to compounds of formula 3 and pharmaceutically acceptable salts thereof.

### FIELD OF THE INVENTION

The present invention relates to novel pregnane-oximino-aminoalkyl-ether compounds. The present invention also relates to process for the synthesis of pregnane-oximino-aminoalkyl-ethers. The present invention further relates to the use of these compounds as antidiabetic and antidyslipidemic agents. More particularly, the invention relates to the synthesis of compounds of formula 3 and their biological profile thereof.

### BACKGROUND OF THE INVENTION

The type II diabetes mellitus accounts for ˜90-95% of all the total cases of diabetics. Epidemiological studies suggest the changed sedentary life style and food habits have immensely contributed towards affliction of the disease to even adult population. The main driving force for the increasing incidence is a staggering increase in obesity, the single most important contributor to the pathogenesis of diabetes mellitus. Prolonged disease condition leads to chronic macro-vascular complications such as retinopathy and nephropathy.

At present, therapy for type II diabetes relies mainly on approaches intended to reduce the hyperglycaemia itself: sulphonylureas which increase insulin secretion from pancreatic beta cells, bi-guanides such as metformin to reduce hepatic glucose production, peroxisome proliferator activated receptors agonists enhancing insulin action and α-glucosidase inhibitors interfering with gut glucose absorption. These therapies however have limited efficacy, limited tolerability and mechanism-based toxicity. Of particular concern is the tendency for most treatments enhance weight gain and also these treatments slowly become refractory over the time and require change of the treatments. These limitations therefore required search of drugs with new mechanism of action.

Bile acids, the metabolites of cholesterol are amphipathic molecules that control the homeostasis of cholesterol, bile acids themselves, lipids and carbohydrates by solubilising and salvaging into the system through a cytosolic nuclear receptor on hepatocytes called FXR (Farnesoid X Receptor) if it is required otherwise excrete them into faeces. The nuclear FXR for bile acids were discovered in 1999 while their homolog membrane receptors for the same ligands, called TGR5 were discovered in 2002. The latter is metabotrophic receptor expressed in adipocytes and myocytes to enhance energy expenditure. The activation of TGR5 is emerging as an attractive target for the treatment of obesity, diabetes, and metabolic syndrome; few examples of TGR5 experimental agonists have been described in literature [Novel, Potent and Selective Bile Acid Derivatives as TGR5 Agonists: Biological Screening, Structure-Activity Relationships, and Molecular Modelling Studies; Hiroyuki Sato et al; J. Med. Chem. 51, 1831 (2008)]. These compounds are of cationic or anionic character in bio-phase as mentioned below:

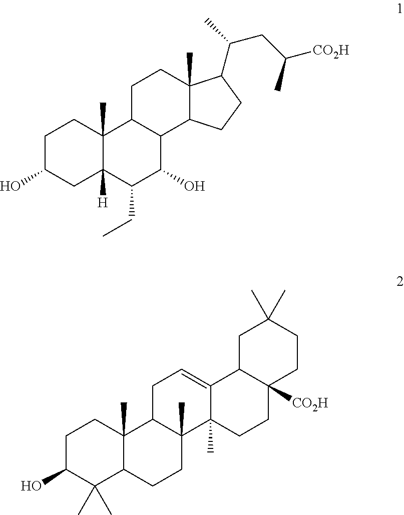

As the TGR5 is a membrane receptor, the compounds likely to stimulate the receptor activity should be of lipophilic with polar groups of anionic or cationic nature distributed around the core skeleton and manifest the action based on the specificity of the compound either for the adipocyte or macrophage receptors. Earlier, we had discovered compound CDRI 80/574 as nuclear FXR antagonist though having no functional group similarity with bile acids but it had significant anti-dyslipidaemic activity [The hypolipidemic natural product guggulsterone acts as an antagonist of the bile acid receptor, Wu et al, Mol. Endocrin. 16, 1590, (2002), Pratap et al U.S. Pat. No. 6,579,862 B1 (2003)].

### OBJECTIVE OF THE INVENTION

The main object of the present invention is to provide novel pregnane compound of formula 3 or a pharmaceutically acceptable salt thereof.

Another object of the present invention is to provide a pharmaceutical composition comprising these pregnane compounds of the present invention with a lipid and sugar lowering property.

Still another object of the present invention is to provide a process for preparation of compounds of formula 3.

Yet another object of the present invention is to provide a method for controlling type II diabetes and associated hyperlipidaemic conditions in mammal by administering a pharmaceutically acceptable amount of formula 3 with or without other-antidiabetic-and-lipid lowering agents.

### SUMMARY OF THE INVENTION

Accordingly, the present invention provides a compound of formula 3 or a pharmaceutically acceptable salt thereof;

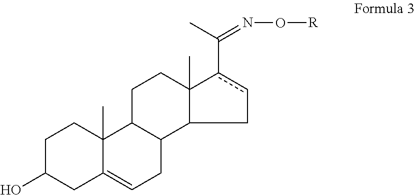

wherein R is selected from the group consisting of hydrogen (H), n-butyl, benzyl and

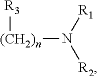

where n is 2 or 3,
R1 and R2 are independently selected from the group consisting of H and an alkyl group or R1 and R2 together form a cyclic system wherein the cyclic system is selected from the group consisting of 4-phenyl-piperazine-1-yl, 4-(2-methoxy phenyl)-piperazinyl, pyrrolidinyl, piperidinyl, azepanyl and morpholine;

### R3 is H or OH,

wherein the alkyl group is selected form the group consisting of ethyl, isopropylamine, diisopropyl and t-butyl amine.

In an embodiment of the invention, the compound is selected from the group consisting of:
3β-Hydroxy-pregna-5,16-dien-20-one-O-n-butyl-oxime (10a),
3β-Hydroxy-pregna-5,16-dien-20-one-O-benzyl-oxime (10b),
3β-Hydroxypregna-5,16-dien-20-one-O-(2-piperidinyl-ethyl)-oxime (11a),
3β-Hydroxypregna-5,16-dien-20-one-O-(2-azepan-1-yl-ethyl)-oxime (11 b),
3β-Hydroxypregna-5,16-dien-20-one-O-(2-morpholin-4-yl-ethyl)-oxime (11c),
3β-Hydroxypregna-5,16-dien-20-one-O-(2-diethylamino-ethyl)-oxime (11d),
3β-Hydroxy-pregna-5,16-dien-20-one-O-[3-{2-hydroxy-3-(4-(2-methoxy-phenyl-piperazinyl)-propyl)}]-oxime (13a),
3β-Hydroxy-pregna-5,16-dien-20-one-O-[3-(2-hydroxy-3-(4-phenyl-piperazinyl)-propyl)]
-oxime (13b),
3β-Hydroxy-pregna-5-en-20-one-O-(2-pyrrolidin-1-yl-ethyl)-oxime (14a),
3β-Hydroxy-pregna-5-en-20-one-O-(2-piperidin-1-yl-ethyl)-oxime (14b),
3β-Hydroxy-pregna-5-en-20-one-O-[2-hydroxy-3-iso-propylamino-propyl)-oxime (16a),
3β-Hydroxy-pregna-5-en-20-one-O[2-hydroxy-3-tert.-butylamino-propyl]-oxime (16b),
3β-Hydroxy-pregna-5-en-20-one-O-[2-hydroxy-3-(4-phenyl-piperazin-1-yl)-propyl]-oxime (16c),
3β-Hydroxy-pregna-5-en-20-one-O-{2-hydroxy-3-[4-(2-methoxyphenyl)-piperzin-1-yl]-propyl}-oxime (16d),
3β-Hydroxy-pregna-5-en-20-one-O-[2-hydroxy-3-(2-pyridyl-piperazin-1-yl)-propyl]-oxime (16f),
3β-Hydroxy-pregna-5-en-2-one-O-[2-hydroxy-3-pipridinyl-propyl)-oxime (16g),
3β-Hydroxy-pregna-5-en-20-one-O-[2-hydroxy-3-(4-methyl-piperazin-1-yl)-propyl]-oxime (16h),
3β-Hydroxy-pregna-5-en-20-one-O-[2-hydroxy-3-diisopropylamino-propyl)-oxime (16i), and
3β-Hydroxy-pregna-5-en-20-one-O-[2-hydroxy-3-diethylamino-propyl)-oxime (16j).

In another embodiment of the invention, the compound is useful for the treatment of diabetes and dyslipidemia

Accordingly the present invention also provides a process for preparation of the compound of formula 3 comprising the steps of:

i. reacting a compound of formula A

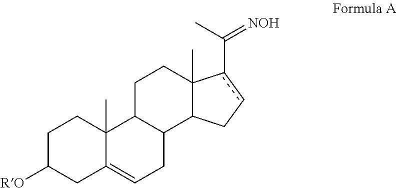

wherein R′ is selected from the group COCH3 or H
with an alkyl halide, a benzyl halide, a substituted aminoethylhalide or an epoxy propylhalide in presence of a base, in a solvent, to obtain a reaction mixture;
ii. evaporating the reaction mixture obtained from step (i) under vacuum, followed by extraction with a water immiscible solvent and purification to obtain corresponding compounds 10(a-b), 11(a-d), 12, 14(a-b) and 15,

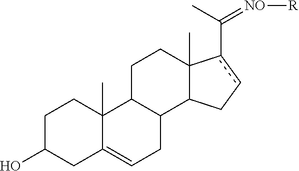

wherein, R is selected from the group consisting of hydrogen (H), n-butyl, benzyl, substituted aminoethyl and epoxy propyl, or cyclic aminoethyl,
10(a-b): butyl or benzyl, 11(a-d): piperidinyl-ethyl or azepan-1-yl-ethyl or morpholin-4-yl-ethyl or diethylamino-ethyl, 12: and 15: epoxypropyl, 14(a-b): pyrrolidin-1-yl-ethyl or piperidin-1-yl-ethyl;
iii. further comprising the step of reacting compound 12 or 15 with an amine obtained from step (ii), in methanol under reflex, followed by purification to obtain compounds 13(a-b) and 16(a-j),

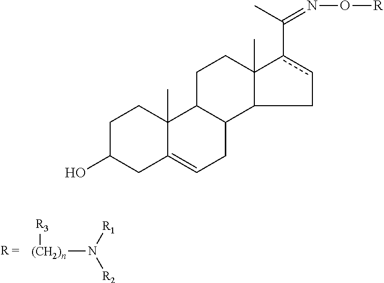

where n is 2 or 3,
R1 and R2 are independently selected from the group consisting of hydrogen (H) and an alkyl group, or R1 and R2 together form a cyclic system wherein the cyclic system is selected from the group consisting of 4-phenyl-piperazine-1-yl, 4-(2-methoxy phenyl)-piperazinyl, pyrrolidinyl, piperidinyl, azepanyl and morpholine,

### R3 is H or OH,

wherein the alkyl group is selected form the group consisting of ethyl, isopropylamine, diisopropyl and t-butyl amine.

In another embodiment of the invention, the water immiscible solvent is selected from the group consisting of chloroform, dichloromethane, ether and ethyl acetate.

In yet another embodiment of the invention, the reaction between the compound of formula A and the alkyl halide, benzyl halide, substituted aminoethylhalide or epoxy propylhalide in step (i) is carried out at a temperature ranging from 30 to 80 degree C. for a period ranging from 10 to 20 hrs.

In yet another embodiment of the invention, the solvent of step (i) is selected from the group consisting of DMF and N-methylpyrrolidone.

In yet another embodiment of the invention, the base of step (i) is selected from the group consisting of sodium hydride and potassium hydride.

In yet another embodiment of the invention the pharmaceutical composition comprising a pharmaceutically effective amount of a compound of formula 3 or a pharmaceutically acceptable salt thereof, optionally along with the carries, diluents and exceipients.

In yet another embodiment of the invention the pharmaceutical composition comprising a pharmaceutically effective amount of a compound of formula 3 or a pharmaceutically acceptable salt thereof, optionally along with pharmaceutically acceptable additives.

In yet another embodiment, said composition is useful for the treatment of diabetes and dyslipidemia.

A method for treating type II diabetes in mammals, comprising the steps of administering to a subject in need thereof, a pharmaceutically effective amount of a compound of formula 3 or a pharmaceutically acceptable salt thereof,

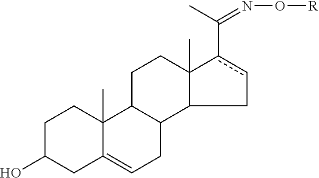

wherein R is selected from the group consisting of hydrogen (H), n-butyl, benzyl and

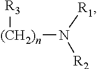

where n is 2 or 3,
R1 and R2 are independently selected from the group consisting of H and an alkyl group, or R1 and R2 together form a cyclic system wherein the cyclic system is selected from the group consisting of 4-phenyl-piperazine-1-yl, 4-(2-methoxy phenyl)-piperazinyl, pyrrolidinyl, piperidinyl, azepanyl and morpholine;

### R3 is H or OH,

wherein the alkyl group is selected form the group consisting of ethyl, isopropylamine, diisopropyl, t-butyl amine
optionally with other antidiabetic and antidyslipidemic agents.

The invention also provides a method for controlling type II diabetes and associated hyperlipidemic conditions in mammals by administering composition containing these derivatives.

Further embodiment of the present invention discloses the compounds, wherein the representative compound is selected from the group consisting of:
3β-Hydroxy-pregna-5,16-dien-20-one-O-n-butyl-oxime (10a)
3β-Hydroxy-pregna-5,16-dien-20-one-O-benzyl-oxime (10b)
3β-Hydroxypregna-5,16-dien-20-one-O-(2-piperidinyl-ethyl)-oxime (11a)
3β-Hydroxypregna-5,16-dien-20-one-O-(2-azepan-1-yl-ethyl)-oxime (11 b)
3β-Hydroxypregna-5,16-dien-20-one-O-(2-morpholin-4-yl-ethyl)-oxime (11c)
3β-Hydroxypregna-5,16-dien-20-one-O-(2-diethylamino-ethyl)-oxime (11d)
3β-Hydroxy-pregna-5,16-dien-20-one-O-{3-{2-hydroxy-3-(4-(2-methoxy-phenyl-piperazinyl)-propyl)}]-oxime (13a)
3β-Hydroxy-pregna-5,16-dien-20-one-O-[3-(2-hydroxy-3-(4-phenyl-piperazinyl)-propyl)]-oxime (13b)
3β-Hydroxy-pregna-5-en-20-one-O-(2-pyrrolidin-1-yl-ethyl)-oxime (14a)
3β-Hydroxy-pregna-5-en-20-one-O-(2-piperidin-1-yl-ethyl)-oxime (14b)
3β-Hydroxy-pregna-5-en-20-one-O-[2-hydroxy-3-iso-propylamino-propyl)-oxime (16a)
3β-Hydroxy-pregna-5-en-20-one-O-[2-hydroxy-3-tert.-butylamino-propyl]-oxime (16b)
3β-Hydroxy-pregna-5-en-20-one-O-[2-hydroxy-3-(4-phenyl-piperazin-1-yl)-propyl]-oxime (16c)
3β-Hydroxy-pregna-5-en-20-one-O-{2-hydroxy-3-(4-(2-methoxyphenyl)-piperzin-1-yl]-propyl}-oxime (16d)
3β-Hydroxy-pregna-5-en-20-one-O-[2-hydroxy-3-(2-pyridyl-piperazin-1-yl)-propyl]-oxime (16f)
3β-Hydroxy-pregna-5-en-2-one-O-[2-hydroxy-3-pipridinyl-propyl]-oxime (16g)
3β-Hydroxy-pregna-5-en-20-one-O-[2-hydroxy-3-(4-methyl-piperazin-1-yl)-propyl]-oxime (16h)
3β-Hydroxy-pregna-5-en-20-one-O-[2-hydroxy-3-diisopropylamino-propyl)-oxime (16i)
3β-Hydroxy-pregna-5-en-20-one-O-[2-hydroxy-3-diethylamino-propyl)-oxime (16j).

Further embodiment of the present invention provides a method for treating type II diabetes in mammals, said method comprising the step of administering a pharmaceutically effective amount of a compound of formula 3 or a pharmaceutically acceptable salt thereof, optionally along with other antidyslipidemic agents.

Furthermore embodiment of the present invention provides a method of treating hyperlipidemic conditions in mammals, said method comprising the step of administering a pharmaceutically effective amount of a compound formula 3, or pharmaceutically acceptable salt thereof, optionally with other antidiabetic and antidyslipidemic agents:

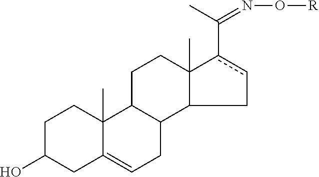

wherein R is selected from the group consisting of hydrogen (H), n-butyl, benzyl and

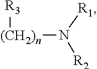

where the number of carbon atoms forming linkage between oximino O and amino N are selected from 2 and 3. Also the three carbon atom linker has hydroxyl functionality. Also NR1R2 is secondary and tertiary amines from group of t-butyl amine, isopropylamine, 4-phenyl-piperazine-1-yl amine, 4-(2-methoxy phenyl)-piperazinyl amine, pyrrolidinyl amine, piperidinyl amine, di-isopropylamine, azepanyl amine and morpholinyl amine.

### ABBREVIATIONS USED

16-DPA=16-Dehydro-pregnelone-acetate; NaH=Sodium hydride; DMF=Dimethylformamide; OGTT=Oral glucose tolerance test; TG=Triglycerides, TC=Total cholesterol, HDL=High density lipoprotein; LDL=Low density lipoprotein; NEFA=Non-esterified fatty acid; PK=Pharmacokinetic; C0=Initial concentration, Cmax=Maximum concentration; T½=Elimination half life time; AUC=Area under curve; Vz=Volume of distribution; F=Bioavailability factor; Cl=clearance; MRT=Mean residence time.

### BRIEF DESCRIPTION OF THE ACCOMPANYING DRAWINGS

FIG. 1: Compounds of the present invention.

FIG. 2: General scheme of preparation of Compounds of the present invention

FIG. 3: Effect of 14b on 2-³H-deoxyglucose uptake by skeletal muscle cell lines.

FIG. 4: Effect of 14b and standard drug Metformin on oral glucose tolerance post sucrose load in normal rats.

FIG. 5: Dose dependent effect of 14b on OGTT of db⁺ mice.

FIG. 6: Blood glucose lowering effect of 14b and standard drug Metformin in sucrose challenged low dosed STZ-induced diabetic rat model.

FIG. 7: Blood glucose lowering effect of 14b and standard drug Metformin in STZ-induced diabetic rats.

FIG. 8: Blood glucose lowering activity of 14b in db/db mice.

FIG. 9: Effect of 14b on serum lipid and serum insulin profile of db/db mice.

FIG. 10: Effect of 14b on body weight of high fructose high fat fed male Syrian golden hamsters.

FIG. 11: Bioavailability data.

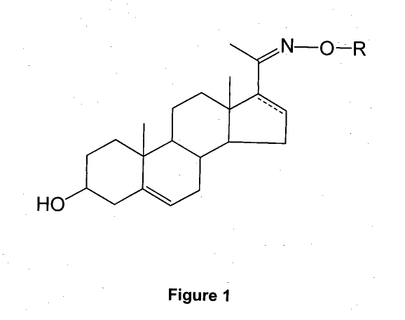

### DETAILED DESCRIPTION OF THE INVENTION

The present invention provides novel pregnane compounds, which exhibits antidiabetic and antidyslipidemic activities in different model systems. More particularly, this invention relates to compounds having the formula 3 and pharmaceutically acceptable salt thereof. Wherein the compounds of formula 3 have 16, 17 olefinic bond or without it on pregnane-oxime-ether as herein after defined.

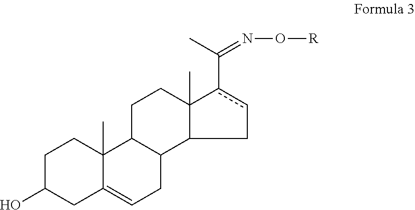

wherein R is selected from the group consisting of hydrogen (H), n-butyl and benzyl,

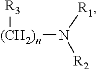

where the number of carbon atoms forming linkage between oximino O and amino N are selected from 2 and 3. Also the three carbon atom linker has hydroxyl functionality. Also NR1R2 is secondary and tertiary amines from group of amines comprising tert-butyl amine, iso-propylamine, 4-phenyl-piperazin-1-yl amine, 4-(2-methoxy-phenyl)-piperazinyl amine, pyrrolidinyl amine, piperidinyl amine, di-iso-propylamine, morpholine amine, azepenyl amine.

The present invention provide a pharmaceutical composition comprising these pregnane compounds with a lipid lowering and sugar lowering property.

The present invention provides a process for preparation of compound of formula 3.

The present invention is to provide a method for controlling type II diabetes and associated hyperlipidemic conditions in mammal by administering a pharmaceutically acceptable amount of formula 3 with or without other antidiabetic and lipid lowering agents.

### Synthesis of Pregnane-Oximino Ether Compounds

The synthesis of the proposed molecules, are given in the Schemes I, II and III bearing the basic functionality with the pregnane skeleton. The synthesis of pregnane-oximes used for the synthesis in these compounds is given in the Scheme-I.

The reaction of 16-dehydropregnelone-acetate (16-DPA) (4) with hydroxylamine hydrochloride in pyridine under cold condition for 72 hrs produced oximino-acetate 5 which was further hydrolyzed to 6 and the latter was used for the condensation with various aminoalkyl halides. The selective hydrogenation of 16, 17 olefinic bond of 16-DPA was accomplished with Pd/C & hydrogen gas by the known method to provide compound 7. The resulting compound was subjected to oximination as mentioned above to yield 8 which was subsequently de-acetylated to 9 (Scheme-I).

The oximino-pregnane 5 was treated with alkyl halides to yield oximino-ether compounds 10 without amino functionality. Compound 6 was further treated with various aminoethyl chlorides to 11. The treatment of 5 with epichlorohydrin followed with basic hydrolysis produced 12. The epoxide 12 on treatment with various amines yielded 13 (Scheme-II). Similarly, the 16,17-dihydro-pregnane-oximino compound 9 was treated with aminoethylchlorides to produce 14 (a, b). The reaction of 9 with epichlorohydrin produced 15 which was again treated with various amines resulted in compounds 16(a-f) (Scheme-III).

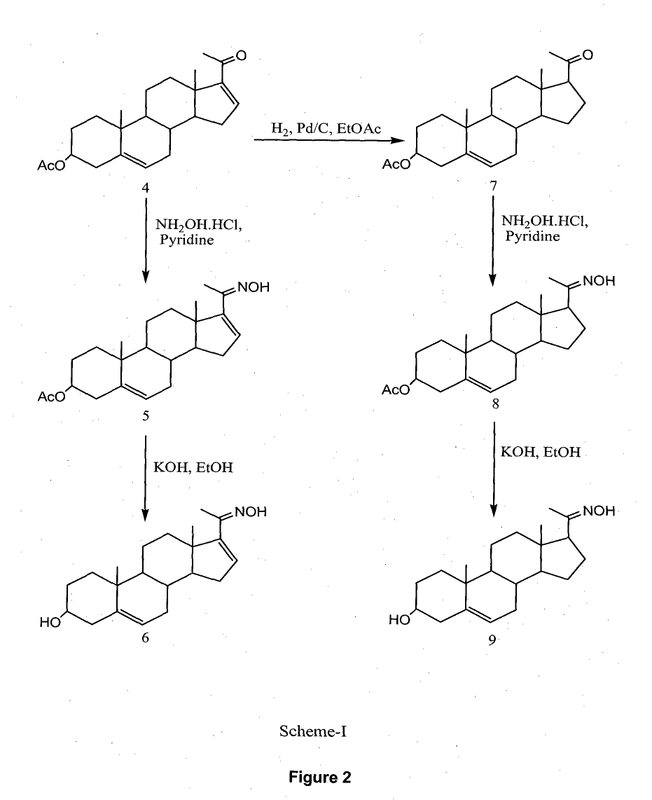

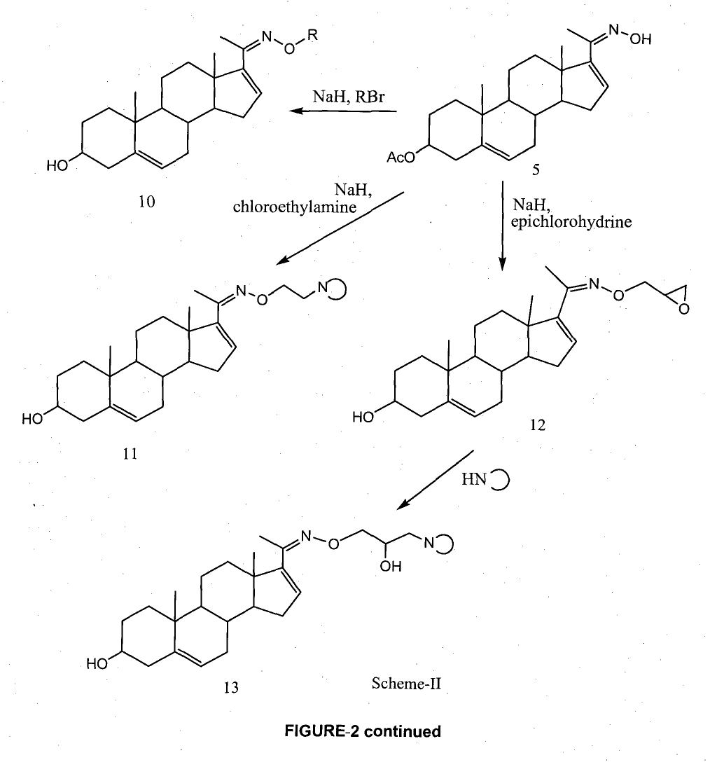

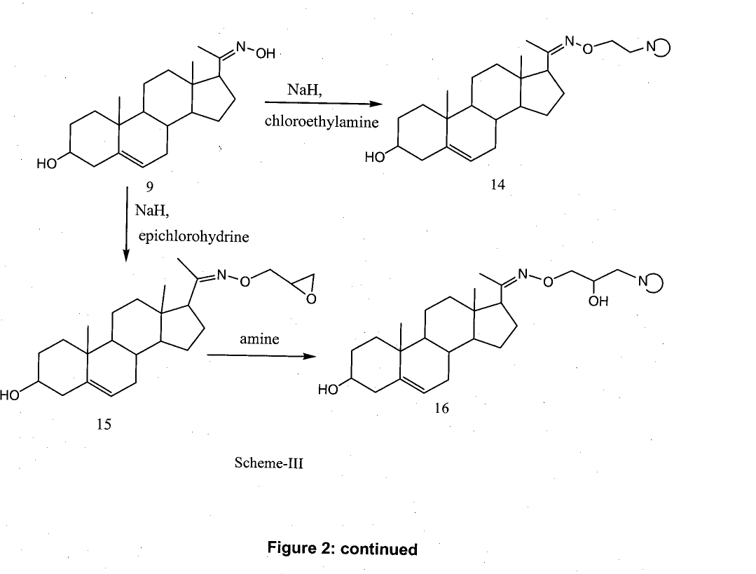

These compounds were then tested for their lipid lowering and antidiabetic activities in various models available with us and results are given in the Tables 1-2. From these studies it emerged that the compounds (10a, 10b) with simple alkyl ether had mainly antidyslipidemic activity while the oximino-ether compounds with amino functionality had both antidyslipidaemic and antidiabetic activities. However, the 16,17-dihydro-oximino-ether compounds (14a, 14b) exhibited better anti-diabetic activity in all the models including db/+ mice models. One of the compounds 14b was tested in detail and the results of antidiabetic and antidyslipidemic activities are mentioned in Table 1-9 and FIG. 2-9. The compound 14b also displayed good bio-availability.

### EXAMPLES

The following examples are given by illustrations and should not be construed to limit the scope of the invention.

### Example 1

### 3β-Hydroxy-pregna-5,16-dien-20-one-oxime (6)

16-DPA (4, 5 gm, 0.014 mole) and hydroxylamine hydrochloride (1.13 g, 0.016 mole) in dry pyridine (100 ml) was stirred until the clear solution appears. It was then left in tightly closed container in refrigerator for four days, poured into water (500 ml) and separated solid collected by filtration, washed with water and dried to afford compound 5 (5 g, 96%). Compound 5 (5 g, 0.013 mole) and 20 mL aq. KOH (5%) in THF (80 mL) was refluxed for 15 hrs. Solvent was removed in vaccuo and the residue extracted with chloroform, washed with water and dried (Na₂SO₄). The solvent was removed and residue crystallized from ethyl acetate to afford 6 (2.9 g, 65%). M. P. 210-12° C.; EIMS: m/z 329 (M⁺); IR (KBr): 3428, 2931, 2836, 1616, 1438 and 1247 cm⁻¹; ¹H NMR (200 MHz, CDCl₃): δ 6.05 (s, 1H, 16-H), 5.36 (d, 1H, 6H, J=4.84 Hz), 3.53-3.49 (m, 1H, 3-H), 2.0 (s, 3H, 21-CH₃), 1.04 (s, 3H, 19-CH₃), 0.95 (s, 3H, 18-CH₃).

Compound 4 was hydrogenated in the presence of H₂, Pd/C to obtain compound 7. Compound 7 was treated with NH2OH.HCl in pyridine to obtain compound 8 (obtained by hydrogenation of 4, 2.0 g, 2.23 mmol. This step needs to be written in the beginning as per the scheme) and aq KOH (2.24 g, 50% solution) in ethanol was refluxed for 5 hr. The solvent was removed and residue taken up in chloroform and washed to give the compound 9. Yield 1.9 g (83.9%); M.P. 188° C.; FABMS: m/z (M+1); IR (KBr): 3433.3, 2930.9, 1688.1, 1539.3, 1355 and 1058.1 cm⁻¹; ¹H NMR (200 MHz, CDCl₃), δ 5.36 (d, 1H, 6-H, J=4.02 Hz), 3.58-3.48 (m, 1H, 3-H), 2.21 (s, 3H, 21-CH₃), 2.03 (s, 3H, 19-CH₃), 0.70 (s, 3H, 18-CH₃).

### Example 2

### 3β-Hydroxy-pregna-5,16-dien-20-one-O-n-butyl-oxime (10a)

Compound 5 (1.75 g, 5 mmol), NaH (2.9 g, 12 mmol) in dry DMF (80 mL) was stirred for one hour at 0° C. To the reaction mixture n-butyl bromide (1.3 mL, 7 mmole) was added and mixture further stirred for 10 hrs at 30 degree C. DMF was evaporated under vaccum. The solid residue was extracted with chloroform and washed with water, dried (Na₂SO₄) and solvent removed. The residue was purified by silica gel column chromatography to afford 10a (1.18 g, 65%). M. p. 170-72° C.; FABMS: m/z 386 (M+1); ¹H NMR (200 MHz, CDCl₃): δ 5.98 (s, 1H, 16-H), 5.37 (d, 1H, 6H, J=4.5 Hz), 3.50-3.45 (m, 1H, 3-H), 4.07 (t, 2H, OCH₂, J=6.52 Hz), 1.93 (s, 3H, 21-CH₃), 1.47-1.33 (m, 4H, —CH₂—CH₂—), 1.05 (s, 3H, 19-CH₃), 0.93 (s, 3H, 18-CH₃), 0.90 (t, 3H, —CH₃, J=7.22 Hz).

### 3β-Hydroxy-pregna-5,16-dien-20-one-O-benzyl-oxime (10b)

Compound 5 (1.13 g, 3 mmol), NaH (2.7 g, 8 mmol) and benzyl bromide (0.6 mL, 5 mmole) in dry DMF (50 mL) were reacted in a similar manner to that described as above to afford 10b, (0.74 g, 58.5%). M. p. 188-90° C.; FABMS: m/z 420 (M+1); ¹H NMR (200 MHz, CDCl₃): δ 7.26 (s, 5H, Ar—H), 6.05 (s, 1H, 16-H), 5.34 (d, 1H, 6H, J=5.58 Hz), 5.32 (s, 2H, —OCH₂—Ar), 3.55-3.50 (m, 1H, 3-H), 2.03 (s, 1H, 21-CH3), 1.04 (s, 3H, 19-CH₃), 0.90 (s, 3H, 18-CH₃).

### 3β-Hydroxy-pregna-5,16-dien-20-one-O-(2-piperidinyl-ethyl)-oxime (11a)

The suspension of 3β-hydroxy-pregna-5,16-dien-20-one-O-oxime (6, 0.6 g, 1.8 mmol) and NaH (0.2 g, 20 mmol) was stirred for 1 hr at 0° C. in DMF. 1-(2-chloro-ethyl)-piperidine hydrochloride (0.33 g, 2 mmol) was added thereafter and the reaction mixture left stirring for another 6 hr at 35° C. DMF was removed under vaccum and the crude product thus obtained was purified by column chromatography to afford compound (11a) yield 0.6 g (68%); m.p. 135-138° C.; FABMS: m/z 441 (M+1); ¹H NMR (200 MHz, CDCl₃): δ 6.00 (s, 1H, 16-H), 5.37 (d, 1H, 6-H, J=2.0 Hz), 4.23 (t, 2H, OCH₂), 3.48 (m, 1H, 3-H), 2.67 (t, 2H, NCH₂), 2.48 (t, 4H, NCH₂), 1.93 (s, 3H, 21-CH₃), 1.489-1.25 (m, 6H, CH₂), 1.05 (s, 3H, 19-CH₃), 0.96 (s, 3H, 18-CH₃).

### 3β-Hydroxypregna-5,16-dien-20-one-O-(2-azepan-1-yl-ethyl)-oxime (11 b)

The suspension of 3β-hydroxy-pregna-5-en-20-one-oxime (10), 1-(2-chloro-ethyl)-azepine hydrochloride in presence of NaH was reacted as above to afford compound 11b, yield 0.6 g (68%); M.P. 127-130° C.; FABMS: m/z 455 (M+1); ¹H NMR (200 MHz, CDCl₃): δ 6.00 (s, 1H, 16-H), 5.37 (d, 1H, 6-H, J=2.0 Hz), 4.23 (t, 2H, OCH₂), 3.48 (m, 1H, 3-H), 2.67 (t, 2H, NCH₂), 2.48 (t, 4H, NCH₂), 1.93 (s, 3H, 21-CH₃), 1.489-1.25 (m, 6H, CH₂), 1.05 (s, 3H, 19-CH₃), 0.96 (s, 3H, 18-CH₃).

### 3β-Hydroxypregna-5,16-dien-20-one-O-(2-morpholin-4-yl-ethyl)-oxime (11c)

The suspension of 3β-hydroxy-pregna-5-en-20-one-oxime (10), 1-(2-chloro-ethyl)-morpholine hydrochloride in presence of NaH was reacted as above to afford compound 11c, yield 0.5 g (67%); M.P. 132-135° C.; FABMS: m/z 444 (M+1); ¹H NMR (200 MHz, CDCl₃), δ 6.04 (s, 1H, 16-H), 5.37 (d, 1H, 6-H, J=4.0 Hz), 4.23 (t, 2H, OCH₂), 3.73 (t, 4H, NCH₂), 2.71 (t, 2H, NCH₂), 2.55 (t, 4H, NCH₂), 1.93 (s, 3H, 21-CH₃), 1.00 (s, 3H, 19-CH₃), 0.96 (s, 3H, 18-CH₃).

### 3β-Hydroxypregna-5,16-dien-20-one-O-(2-diethylamino-ethyl)-oxime (11d)

The suspension of 3β-hydroxy-pregna-5-en-20-one-oxime (10), 1-(2-chloro-ethyl)-diethylamine hydrochloride in presence of NaH (0.2 g, 20 mmol) was reacted as above to afford compound 11d, yield 0.62 g (69%); M.P. 156-160° C.; FABMS: m/z 429 (M+1); ¹H NMR (200 MHz, CDCl₃), δ 6.00 (s, 1H, 16-H), 5.36 (s, 1H, 6-H), 4.19 (t, 2H, OCH₂), 3.31 (m, 1H, 3-H), 2.80 (t, 2H, NCH₂), 2.65-2.58 (q, 6H, NCH₂CH₃), 1.93 (s, 3H, 21-CH₃), 1.05 (s, 3H, 19-CH₃), 0.63 (s, 3H, 18-CH₃).

### 3β-Hydroxy-pregna-5,16-dien-20-one-O-(2,3-epoxypropyl)-oxime (12)

Compound 5 (1.13 g, 3 mmol), NaH (2.16 g, 9 mmol) in dry DMF (100 ml) was stirred for an hour at 0° C. and epichlorohydrin (1.17 ml, 15 mmol) was then added drop wise. The reaction mixture was stirred over night at 28 degree C. DMF removed under vacuum. Residue was extracted with chloroform and washed with water, dried over Na₂SO₄ and solvent removed in vacuo and the crude was purified by column chromatography to afford compound (12); yield 6.2 g (75.7%); m. p. 104° C.; FABMS: m/z 386 (M+1); ¹H NMR (200 MHz, CDCl₃), δ 6.17 (s, 1H, 16-H), 5.37 (d, 1H, 6-H, J=4.32 Hz), 4.35-4.19 (m, 2H, —OCH₂), 3.66-3.43 (m, 2H, —CH₂—O), 3.0 (m, 1H, —CH—OH), 2.2 (s, 3H, 21-CH₃), 1.25, 1.04 (2s, 6H, 19&18-CH₃).

### 3β-Hydroxy-pregna-5,16-dien-20-one-O-[2-hydroxy-3-(2-hydroxy-3-(4-phenylpiperazinyl)-propyl)]-oxime (13a)

The solution of 3β-hydroxy-pregna-5-en-2-one-O-(2,3-epoxypropyl)-oxime (12, 0.74 g, 2 mmol), 4-N-phenylpiperazine (0.3 mL, 3 mmol) in dry methanol (40 ml) was refluxed for 6 hr. The methanol was evaporated in vaccuo. The resultant residue was purified by column chromatography to give compound 13a, yield 1.5 g (51%); M.P. 127° C.; FABMS: m/z 548 (M+1); ¹H NMR (200 MHz, CDCl₃): δ 7.29 (d, 2H, Ar—H, J=8.42), 6.94-6.82 (m, 3H, ArH), 6.05 (s, 1H, 16-H), 5.37 (d, 1H, 6-H, J=4.64 Hz), 4.13-4.408 (m, 3H, ═N—O—CH₂—CH—OH), 3.52-3.49 (m, 1H, 3-H), 3.24-3.19 (m, 2H, NCH₂), 2.52-2.49 (m, 4H, NCH₂), 1.98 (s, 3H, 21-CH₃), 1.04 (s, 3H, 19-CH₃), 0.95 (s, 3H, 18-CH₃).

### 3β-Hydroxy-pregna-5,16-dien-20-one-O-[2-hydroxy-3-{2-hydroxy-3-(4-(2-methoxyphenylpiperazinyl)-propyl)]-oxime (13b)

The reaction of 3β-hydroxy-pregna-5-en-2-one-O-(2,3-epoxypropyl)-oxime (12), 4-N-2-methoxy-phenylpiperazine as above produced 13b, yield (48.5%); M.P. 155° C.; FABMS: m/z 578 (M+1); ¹H NMR (200 MHz, CDCl₃): δ 7.04-6.85 (d, 4H, Ar—H), 6.08 (s, 1H, 16-H), 5.36 (d, 1H, 6-H, J=4.4 Hz), 4.13-4.408 (m, 3H, ═N—O—CH₂—CH—OH), 3.86 (s, 3H, OCH3), 3.52-3.49 (m, 1H, 3-H), 3.14-3.09 (m, 2H, Ar—NCH₂), 2.92-2.86 (m, 2H, NCH₂), 2.70-2.60 (m, 4H, NCH2), 1.87 (s, 3H, 21-CH₃), 1.04 (s, 3H, 19-CH₃), 0.94 (s, 3H, 18-CH₃).

### Example 3

### 3β-Hydroxy-pregna-5-en-20-one-O-(2-pyrrolidin-1-yl-ethyl)-oxime (14a)

The suspension of 3β-hydroxypregna-5-en-20-one-oxime (9, 0.6 g, 1.8 mmol) and NaH (0.2 g, 20 mmol) in DMF was stirred for 1 hr at 0° C. 1-(2-chloro-ethyl)-pyrrolidine hydrochloride (0.33 g, 2 mmol) was added there after and the reaction mixture left stirring for another 6 hr at 35° C. DMF was removed under vaccum and the crude product thus obtained was purified by column chromatography to afford compound 14a, yield 0.6 g (68%); M.P. 140-142° C.; FABMS: m/z 429 (M+1); ¹H NMR (200 MHz, CDCl₃): δ 5.36 (d, 1H, 6-H, J=3.6 Hz), 4.28 (t, 2H, OCH₂), 3.53-3.48 (m, 1H, 3-H), 2.96 (t, 2H, NCH₂), 1.81 (s, 3H, 21-CH₃), 1.29-1.20 (q, 4H, CH₂), 1.01 (s, 3H, 19-CH₃), 0.62 (s, 3H, 18-CH₃).

### 3β-Hydroxy-pregna-5-en-20-one-O-(2-piperidin-1-yl-ethyl)-oxime (14b)

The suspension of 3β-hydroxy-pregna-5-en-20-one-oxime (9, 0.6 g, 1.8 mmol), 1-(2-Chloro-ethyl)-piperidine hydrochloride (0.33 g, 2 mmol) in presence of NaH (0.2 g, 20 mmol) was reacted as above to afford compound 14b; yield 0.5 g (67%); M.P. 130-135° C.; FABMS: m/z 443 (M+1); ¹H NMR (200 MHz, CDCl₃): δ 5.36 (d, 1H, 6-H, J=6.0 Hz), 4.18 (t, 2H, OCH₂), 3.50-3.49 (m; 1H, 3-H), 2.48 (t, 2H, NCH₂), 1.81 (s, 3H, 21-CH₃), 1.67-1.42 (m, 4H, CH₂), 1.01 (s, 3H, 19-CH₃), 0.62 (s, 3H, 18-CH₃).

### 3β-Hydroxy-pregna-5-en-20-one-O-(2,3-epoxypropyl)-oxime (15)

Suspension of compound 9 (7.0 g, 21.0 mmol), NaH (2.04 g, 85 mmol) in dry DMF (100 ml) was stirred for an hour at 0° C. and epichlorohydrin (6.0 ml, 126 mmol) was then added drop wise. The reaction mixture was stirred over night at 31 degree C. DMF removed under vacuum. Residue was extracted with chloroform and washed with water, dried over Na₂SO₄ and solvent removed in vaccuo and the crude was purified by column chromatography to afford compound 15; yield 6.2 g (75.7%); M.P. 110° C.; FABMS: m/z 388 (M+1); ¹H NMR (200 MHz, CDCl₃), δ 5.37 (d, 1H, 6-H, J=4.32 Hz), 4.35 (dd, 1H, J=11.1 Hz and 4.2 Hz), 4.19 (dd, 1H, J=12.75 and 6.6 Hz), 3.66-3.63 (m, 1H, —CH—OH), 3.42-3.29 (m, 1H), 2.97 (t, 1H, J=6.0 Hz), 2.81-2.76 (m, 1H), 1.97 (s, 3H, 21-CH₃), 1.04 (s, 3H, 19-CH₃).

### 3β-Hydroxy-pregna-5-en-20-one-O-[2-hydroxy-3-tert.-butylamino-propyl]-oxime (16a)

The solution of 3β-hydroxy-pregna-5-en-2-one-O-(2,3-epoxypropyl)-oxime (15, 0.5 g, 1.3 mmol), tert. butylamine (0.3 ml, 3.2 mmol) in dry methanol (60 ml) was refluxed for 5 hr. The methanol was evaporated in vaccuo. The resultant residue was purified by column chromatography to give compound 16a. Yield 0.5 g (56.75%); M. P. >200° C.; FABMS: m/z 461 (M+1); ¹H NMR (200 MHz, CDCl₃): δ 5.36 (s, 1H, 6-H), 4.12-4.07 (m, 3H, OCH₂CHOH), 3.39-3.38 (m, 1H, CHOH, 3-H), 2.59-2.58 (m, 2H, NH—CH₂), 1.83 (s, 3H, 21-CH₃), 1.36 (s, 9H, C{CH₃}₃), 1.00 (s, 3H, 19-CH₃), 0.62 (s, 3H, 18-CH₃).

### 3β-Hydroxy-pregna-5-en-20-one-O-[2-hydroxy-3-iso-propylamino-Propyl)-oxime (16b)

The reaction of 3β-hydroxy-pregna-5-en-2-one-O-(2,3-epoxypropyl)-oxime (15, 0.5 g, 1.3 mmol) and iso-propylamine (0.33 ml, 3.2 mmol) in dry methanol (50 ml) under reflux as above furnished 16b, yield 0.5 g (54.3%); M.P. (hygroscopic); FABMS: m/z 447 (M+1); ¹H NMR (200 MHz, CDCl₃) δ 5.36 (d, 1H, 6-H, J=4.4 Hz), 4.12 (d, 2H, OCH₂, J=4.84 Hz), 3.94-3.88 (m, 1H, —CHOH—), 3.55-3.31 (m, 1H, 3-H), 2.89-2.75 (m, 3H, CH₂—NH—CH), 1.95 (s, 3H, 21-CH₃), 1.42-1.34 (m, 6H, CH{CH₃}₂), 1.04 (s, 3H, 19-CH₃), 0.95 (s, 3H, 18-CH₃).

### 3β-Hydroxy-pregna-5-en-20-one-O-[2-hydroxy-3-(4-phenyl-piperazin-1-yl)-propyl]-oxime (16c)

The reaction of 3β-hydroxy-pregna-5-en-20-one-O-(2,3-epoxypropyl)-oxime (15, 700 mg, 1.8 mmol) and 4-N-phenylpiperazine (0.5 ml, 3.6 mmol) in dry methanol (40 ml) as above afforded 16c; yield 0.9 g (51%); M.P. 180° C.; FABMS: m/z 550 (M+1); ¹H NMR (200 MHz, CDCl₃) δ 7.30-7.23 (m, 2H, ArH), 6.94-6.82 (m, 3H, Ar—H), 5.36 (d, 1H, 6-H, J=4.0 Hz), 4.09-4.08 (m, 3H, ═N—OCH₂—CH—OH), 3.52-3.49 (m, 1H, 3-H), 3.21-3.19 (m, 4H, Ar—NCH₂), 2.95-2.86 (m, 2H, N—CH₂), 2.65-2.59 (m, 4H, NCH₂), 1.85 (s, 3H, 21-CH₃), 1.01 (s, 3H, 19-CH₃), 0.64 (s, 3H, 19-CH₃).

### 3β-Hydroxy-pregna-5-en-20-one-O-{2-hydroxy-3-[4-(2-methoxyphenyl)-Piperzin-1-yl]-propyl}-oxime (16d)

1-(2-Methoxy-phenyl)-piperazine hydrochloride salt (1.0 g, 1.8 mmol) was dissolved in aqueous NaOH solution and extracted with ether, with water, dried over Na₂SO₄. Removal of the solvent provided acid free amine. The reaction of free amine and 3β-hydroxy-pregna-5-en-20-one-O-(2,3-epoxypropyl)-oxime (15) as above afforded the compound 16d. Yield 0.85 g (48.5%); M.P. 145-149° C.; FABMS: m/z 580 (M+1); ¹H NMR (200 MHz, CDCl₃): δ 7.01-6.84 (m, 4H, Ar—H), 5.36 (d, 1H, 6-H, J=6.0 Hz), 4.09 (s, 3H, ═N—OCH₂—CH—OH), 3.86 (s, 3H, Ar—OCH₃), 3.52-3.49 (m, 1H, 3-H), 3.10 (s, 4H, Ar—NCH₂), 2.92-2.86 (m, 2H, N—CH₂), 2.70-2.60 (m, 4H, NCH₂), 1.85 (s, 3H, 21-CH₃), 1.01 (s, 3H, 19-CH₃), 0.64 (s, 3H, 18-CH₃).

### 3β-Hydroxy-pregna-5-en-20-one-O-[2-hydroxy-3-(2-pyridyl-piperazin-1-yl)-propyl]-oxime (16e)

The reaction of 3β-hydroxy-pregna-5-en-20-one-O-(2,3-epoxypropyl)-oxime (15, 700 mg, 1.8 mmol) and 2-pyridyl-piperazine (0.6 ml, 3.6 mmol) in dry methanol (40 ml) as above afforded compound 16e; yield 0.9 g (51%); M.P. 160° C.; FABMS: m/z 580 (M+1); ¹H NMR (200 MHz, CDCl₃): δ 8.17 (s, 1H, ArH), 7.47 (t, 1H, ArH), 6.64 (d, 2H, ArH), 5.37 (d, 1H, 6-H, J=4.64 Hz), 4.09 (m, 3H, ═N—OCH₂—), 3.55 (s, 1H, 3-H, CH₂—CH—OH), 2.70-2.48 (m, 4H, NCH₂), 2.27-2.17 (m, 4H, N—CH₂), 1.85 (s, 3H, 21-CH₃), 1.01 (s, 3H, 19-CH₃), 0.63 (s, 3H, 18-CH₃).

### 3β-Hydroxy-pregna-5-en-2-one-O-[2-hydroxy-3-pipridinyl-propyl)-oxime (16f)

The reaction of 3β-hydroxy-pregna-5-en-20-one-O-(2,3-epoxypropyl)-oxime (15, 1.0 g, 2.5 mmol) and cyclohexyl amine (0.6 ml, 5.0 mmol) as above afforded compound 16f; yield 0.7 g (54.7%); M.P. 110° C.; FABMS: m/z 473 (M+1); ¹H NMR (200 MHz, CDCl₃): δ 5.35 (d, 1H, 6-H, J=6.0 Hz), 4.67 (s, 3H, OCH₂CHOH), 3.34-3.60 (m, 1H, 3-H), 2.96-2.93 (m, 6H, NCH₂), 2.02 (t, 4H, NCH₂), 1.82 (s, 3H, 21-CH₃), 1.54 (t, 6H), 1.04 (s, 3H, 19-CH₃), 0.61 (s, 3H, 18-CH₃).

### 3β-Hydroxy-pregna-5-en-20-one-O-[2-hydroxy-3-(4-methyl-piperazin-1-yl)-propyl]-oxime (16g)

The reaction of 3β-hydroxy-pregna-5-en-20-one-O-(2,3-epoxypropyl)-oxime (15, 1.0 g, 2.5 mmol) and 4-methyl-piperazine (0.6 ml, 5.0 mmol) as above afforded compound 16g; yield 0.9 g (51%); M. P. 100° C.; FABMS: m/z 488 (M+1); IR (KBr): 3397, 2934, 1460 cm⁻¹; ¹H NMR (200 MHz, CDCl₃): δ 5.36 (d, 1H, 6-H, J=4.4 Hz), 4.05 (s, 3H, OCH₂—CH{OH}), 3.49 (m, 1H, 3-H), 2.45 (t, 8H, NCH₂) 2.30 (s, 3H, NCH₃), 1.84 (s, 3H, 21-CH₃), 1.14 (s, 3H, 19-CH₃), 0.62 (s, 3H, 18-CH₃).

### 3β-Hydroxy-pregna-5-en-20-one-O-[2-hydroxy-3-diethylamino-propyl)-oxime (16h)

The reaction of 3β-hydroxy-pregna-5-en-20-one-O-(2,3-epoxypropyl)-oxime (15, 1.0 g, 2.5 mmol) and diethylamine (0.6 ml, 5.0 mmol) as above afforded compound 16h; yield 0.7 g (54.5%); M.P. 157-159° C.; FABMS: m/z 433 (M+1); ¹H NMR (200 MHz, CDCl₃): δ 5.36 (d, 1H, 6-H, J=4.0 Hz), 4.23-4.03 (m, 2H, OCH₂CHOH), 3.34-3.48 (m, 1H, 3-H), 3.00-2.83 (q, 4H, CH₂), 1.84 (s, 3H, 21-CH₃), 1.24 (t, 6H, CH₃), 1.09 (s, 3H, 19-CH₃), 0.62 (s, 3H, 18-CH₃).

### 3β-Hydroxy-pregna-5-en-20-one-O-[2-hydroxy-3-diisopropylamino-propyl)-oxime (16i)

The reaction of 3β-hydroxy-pregna-5-en-20-one-O-(2,3-epoxypropyl)-oxime (15, 1.0 g, 2.5 mmol) and diisopropyl amine (0.8 ml, 5.0 mmol) in dry methanol (50 ml) as above to afford compound 16i; yield 0.7 g (53.7%); M.P. 135-139° C.; FABMS: m/z 476 (M+1); ¹H NMR (200 MHz, CDCl₃); δ 5.35 (d, 1H, 6-H, J=6.0 Hz), 4.22-4.13 (m, 2H, OCH₂CHOH), 3.98-3.41 (m, 1H, 3-H), 2.27 (s, 2H, CH), 1.86 (s, 3H, 21-CH₃), 1.12 (t, 12H, CH₃), 1.04 (s, 3H, 19-CH₃), 0.95 (s, 3H, 18-CH₃).

### Example 4

### Evaluation of antidyslipidaemic activity in Triton-Rat Model

The compounds so far synthesized were evaluated for their antidyslipidaemic activity in Triton-induced hyperlipidemic rat model.

### Triton Model

Male charles foster rats weighing around 200 to 225 g were divided into control, dyslipidemic and dyslipidemic plus drug treated groups containing six animals in each group. Dyslipidemia was induced by administration of triton WR-1339 (200 mg/kg intraperitoneally). All animals were maintained on a special pellet diet and water ad libitum. Test compounds and standard drug (Gemfibrozil) were macerated in 0.2% aqueous gum acacia. The fine suspension of test compounds and standard drug were fed orally to the respective groups at the dose of 100 mg/kg simultaneously with triton WR-1339 in drug treated group. The animals of control group received the same amount of gum acacia by similar route of administration. At the end of the experiment i.e. after 18 h, blood was withdrawn from retro orbital plexus and plasma separated and was used for the assay of total cholesterol, phospholipids and triglycerides (Table 1).

### Lipid Estimation

### Cholesterol

Total plasma cholesterol was estimated using the kit and instructions as provided by the manufacturer Roche Diagnostics. Cholesterol esters are enzymatically hydrolyzed by cholesterol esterase (CE) to cholesterol and free fatty acids. Free cholesterol, including that originally present, is then oxidized by cholesterol oxidase (CO) to cholest-4-en-3-one and hydrogen peroxide. The hydrogen peroxide combines with hydroxybenzoic acid (HBA) and 4-aminoantipyrine (4-AAA) in the presence of peroxidase (POD) to form a red chromophore (quinoneimine) which is quantitated at 500-505 nm. The intensity of red color formed is directly proportional to the concentration of total cholesterol present in the plasma. The result of the experiment is discussed in Table 1.

### Triglycerides

Total plasma triglycerides were estimated using the kit and instructions as provided by the manufacture i.e. Roche Diagnostics. Lipoprotein lipase hydrolyses triglycerides to yield glycerol and fatty acids. Glycerol kinase converts glycerol to glycerol-3-phosphate, which is oxidized by glycerol phosphate oxidase to form dihydroxy-acetone phosphate and hydrogen peroxide. In the presence of peroxidase, hydrogen peroxide oxidatively couples with 4-aminoantipyrine and 4-chlorophenol to produce red chromophore quinonimine which is quantitated at 500-505 nm. The intensity of red color formed is directly proportional to the concentration of triglycerides present in the plasma. The result of the experiment is discussed in Table 1.

### Phospholipids

Plasma (0.2 ml) was digested with perchloric acid (1.0 mL) at 180° C. for 1-1.5 h till the solution became colorless. The liberated inorganic phosphate (Pi) was measured by the method of Fiske and Subbarow. 1 ml of 2.5% ammonium molybdate (prepared in 5N sulphuric acid) and 0.5 ml reducing agent (4-amino naphthol sulphonic acid, 0.2%), sodium metabisulphite (2.4% w/v in distilled water) was added to the above tubes and mixed well. The reaction mixture was distilled with 2.5 ml of triple distilled water and kept at 60° C. in water bath for 20 min. For standard, an appropriate amount of potassium dihydrogen phosphate dissolved in triple distilled water containing 2 to 10 μg phosphorous (Pi) was run simultaneously with the experimental tubes. The optical density of the blue color was recorded at 620 nm against reagent blank. The values of Pi were converted into phospholipids by multiplying with 25 (a constant calibrated from Pi value of lecithin). The result of the experiment is discussed in Table 1.

TABLE 1
Effect of test compound on serum lipid profile of Triton-induced hyperlipidemic rat model

| | | % change in lipid | | |
|---|---|---|---|---|
| Test | | profile | | |
| compound | Structure | TC | PL | TG |
| 6 | 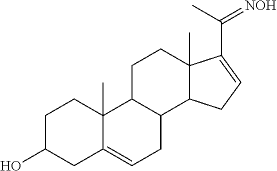 | 24 | 34 | 33 |
| 10a | 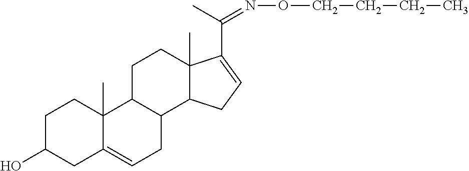 [SMILES: CCCCO/N=C(/C)C1=CCC2C3CC=C4CC(O)CCC4(C)C3CCC12C] | 39 | 31 | 47 |
| 10b | 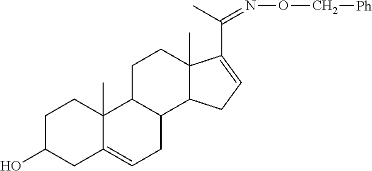 [SMILES: C/C(=N/OCc1ccccc1)C1=CCC2C3CC=C4CC(O)CCC4(C)C3CCC12C] | 40 | 32 | 39 |
| 13a | 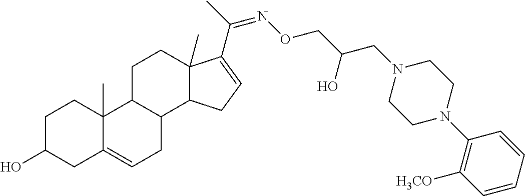 [SMILES: COc1ccccc1N1CCN(CC(O)CO/N=C(/C)C2=CCC3C4CC=C5CC(O)CCC5(C)C4CCC23C)CC1] | 32 | 27 | 4.0 |
| 13b | 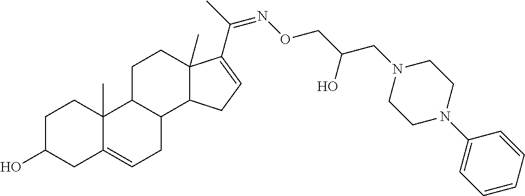 [SMILES: C/C(=N/OCC(O)CN1CCN(c2ccccc2)CC1)C1=CCC2C3CC=C4CC(O)CCC4(C)C3CCC12C] | 21 | 18 | 4.0 |
| 9 | 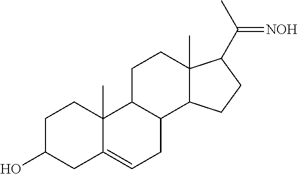 | 18 | 22 | 20 |
| 16a | 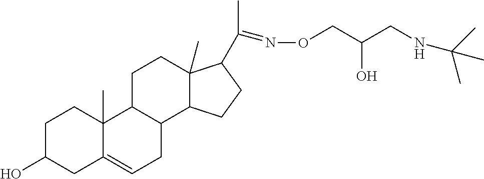 [SMILES: C/C(=N\OCC(O)CNC(C)(C)C)C1CCC2C3CC=C4CC(O)CCC4(C)C3CCC12C] | 14 | 16 | 14 |
| 16b | 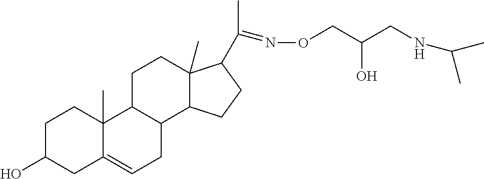 [SMILES: C/C(=N\OCC(O)CNC(C)C)C1CCC2C3CC=C4CC(O)CCC4(C)C3CCC12C] | 23 | 24 | 22 |
| 16c | 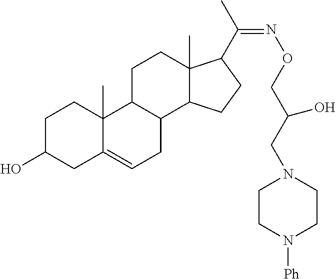 [SMILES: C/C(=N/OCC(O)CN1CCN(c2ccccc2)CC1)C1CCC2C3CC=C4CC(O)CCC4(C)C3CCC12C] | 27 | 25 | 21 |
| 16d | 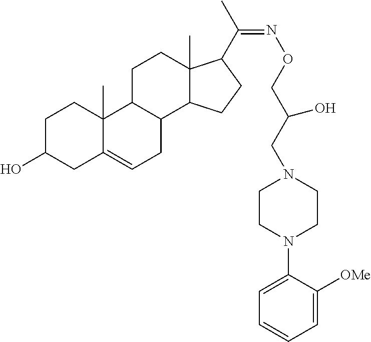 [SMILES: COc1ccccc1N1CCN(CC(O)CO/N=C(/C)C2CCC3C4CC=C5CC(O)CCC5(C)C4CCC23C)CC1] | 27 | 25 | 21 |
| 16f | 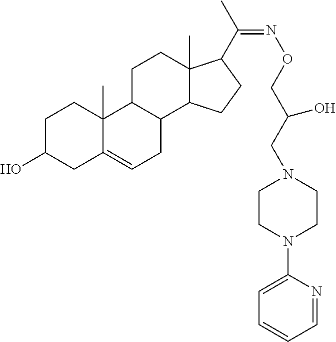 [SMILES: C/C(=N/OCC(O)CN1CCN(c2ccccn2)CC1)C1CCC2C3CC=C4CC(O)CCC4(C)C3CCC12C] | 23 | 21 | 24 |
| 16g | 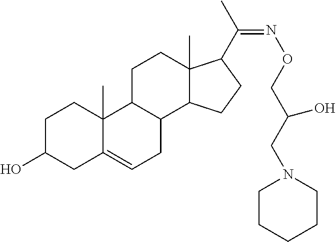 [SMILES: C/C(=N/OCC(O)CN1CCCCC1)C1CCC2C3CC=C4CC(O)CCC4(C)C3CCC12C] | 18 | 20 | 19 |
| 16h | 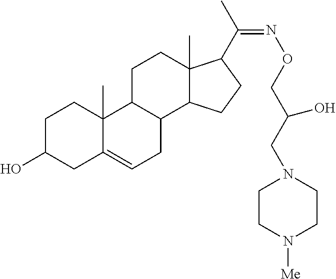 [SMILES: C/C(=N/OCC(O)CN1CCN(C)CC1)C1CCC2C3CC=C4CC(O)CCC4(C)C3CCC12C] | 22 | 22 | 20 |
| 16i | 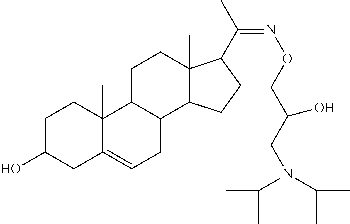 [SMILES: C/C(=N/OCC(O)CN(C(C)C)C(C)C)C1CCC2C3CC=C4CC(O)CCC4(C)C3CCC12C] | 16 | 18 | 22 |
| 16j | 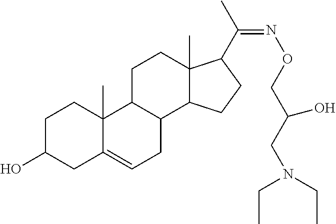 [SMILES: CCN(CC)CC(O)CO/N=C(/C)C1CCC2C3CC=C4CC(O)CCC4(C)C3CCC12C] | 13 | 17 | 15 |
| Standard | Gemfibrozil | 38 | 26 | 26 |

### Example 5

### Evaluation of Antihyperglycaemic Activity

### A. In Vitro Antihyperglycaemic Activity Evaluation Employing 2-³H-Deoxyglucose Uptake by Skeletal Muscle Cells

The fold increase in 2-deoxy-glucose uptake in the presence of test compound by rat skeletal muscle cells (L-6 myotubes) was considered as % antihyperglycemic activity of the test compound which was carried out as follows. L6 myotubes were incubated with increasing concentrations of 14b and the standard drug rosiglitazone (10 μM) for 16 h. After incubation glucose uptake was assessed for 5 min in HEPES-buffered saline [140 mM NaCl, 20 mM HEPES, 5 mM KCl, 2.5 mM MgSO4, 1 mM CaCl2 (pH 7.4)] containing 10 μM 2-deoxyglucose (0.5 μCi/ml 2-[³H]-deoxyglucose) at room temperature. Subsequently cells were rinsed with an ice-cold solution containing 0.9% NaCl and 20 mM D-glucose. To quantify the radioactivity incorporated, cells were lysed with 0.05 N NaOH and lysates were counted with scintillation fluid in a β-counter. Nonspecific uptake was determined in the presence of cytochalasin B (50 μM) during the assay, and these values were subtracted from all other values. Glucose uptake was measured in triplicate and normalized to total protein, was expressed as fold increase with respect to control cells.

### Results Showing Effect of 14b on 2-³H-Deoxyglucose Uptake by Skeletal Muscle Cell Lines

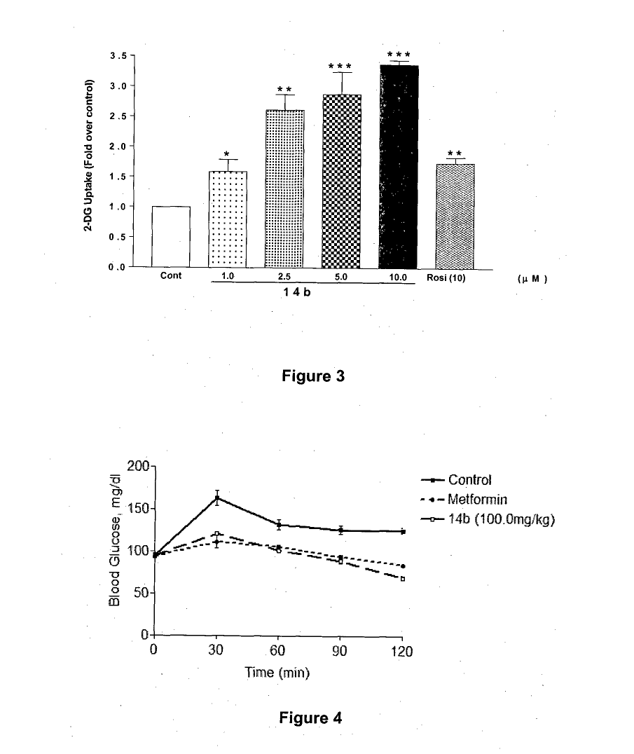

As shown in FIG. 3, incubation of L6 myotubes with 14b increased glucose uptake in a concentration-dependent manner. 14b increased glucose uptake in L6 myotubes to a significant level at a minimum concentration of 1.0 μM (p<0.05) while maximal stimulation was observed at 10 μM (3.37±0.12-fold, p<0.001 vs. control). 14b stimulated glucose uptake by 1.59±0.28, 2.61±0.45, and 2.88±0.74 folds vs. control at 1.0 μM, 2.5 μM and 5.0 μM concentrations, respectively. Rosiglitazone was used as positive control which caused 1.74±0.19-fold stimulation (p<0.01, vs. control) of glucose uptake at 10 μM concentration under identical experimental conditions. Glucose uptake was completely blocked in presence of cytochalasin-B (50 μM), added to transport solution, suggesting the involvement of glucose transporter mediated uptake in response to 14b.

### FIG. 3: Effect of 14b on 2-³H-Deoxyglucose Uptake by Skeletal Muscle Cell Lines

### B. In Vivo Antihyperglycaemic Effect

i. Sucrose Loaded Rat Model

Male albino rats (Sprague Dawley Strain) of body weight 160±20 were procured from the colony of CDRI animal house. 5 to 6 animals were kept in one polypropylene cage and acclimatized for one week in the new environment. On the day of experiment blood glucose of all the overnight fasted animals was checked by Glucostrips (Roche) using Glucometer (ACCU-CHEK II; Roche Diagnostics, USA). Rats or mice having blood glucose levels between 65-80 mg/dl were finally included in the experiment. Animals were divided into groups consisting of five animals in each. Group one was considered as a control group which receives vehicle 1% gum acacia, where as the other groups were termed as experimental groups and were given the desired doses of the test compounds and standard drug (Metformin), respectively. The test compounds and standard drug was always prepared in the vehicle 1% gum acacia. An oral glucose load of 3.0 g/kg was given to the animals of all the groups 30 min post administration of the test samples/vehicle/standard drug. Blood glucose levels of the animals of all the groups were again measured at 30, 60, 90 and 120 min post sucrose load. Food (not water) was removed from the cages during the experimental period. The improvement in oral sucrose tolerance (OGTT) was determined by plotting a graph between time post glucose load and blood glucose levels on x and y axis, respectively and determining the area under curve (AUC) between 0 to 120 min were calculated of each group. Comparing the AUC of test sample or drug treated groups compared to control group determined the percent improvement in OGTT by test sample and standard drugs and termed as antihyperglycaemic activity. Statistical analysis between the groups was done by employing Students t test

### Antihyperglycaemic Activity in Sucrose Loaded Rat Model

Table 3 and FIG. 4, show the effect of 14b and standard drug Metformin on oral sucrose tolerance post sucrose load in rats. 14b and standard drug Metformin showed around 26.0% (p<0.01) and 24.4% (p<0.001) improvement in OGTT post sucrose load, respectively in the normal rats.

TABLE 3
Antihyperglycaemic effect of 14b and standard drug Metformin in sucrose
loaded rat model.

| | Blood glucose profile (mg/dl) | | | | | % |
|---|---|---|---|---|---|---|
| Treatment | min post glucose load | | | | | Antihyperglycaemic |
| Group | 0 | 30 | 60 | 90 | 120 | activity |
| Control | 95.4 ± 3.0 | 163.2 ± 9.2 | 132.0 ± 6.3 | 126.2 ± 4.9 | 125.2 ± 3.4 | |
| 14b | 94.2 ± 2.6 | 120.8 ± 2.1 | 101.4 ± 2.9 | 89.4 ± 3.3 | 69.4 ± 2.3 | 26.0** |
| treated | | | | | | |
| (100 mg/kg) | | | | | | |
| Metformin | 94.8 ± 2.1 | 111.4 ± 7.8 | 105.8 ± 2.1 | 94.0 ± 2.0 | 84.2 ± 1.3 | 24.4*** |
| treated | | | | | | |
| (100 mg/kg) | | | | | | |

Statistical significance *p < 0.05, **p < 0.01 and ***p < 0.001

ii. Glucose Loaded Db/+Mice

Male C57BL6-db/+ mice (12-18 week old, 18-20 g body weight) were procured from National Animal Laboratory Centre (NALC) of Central Drug Research Institute (CDRI), Lucknow, India Animals were housed in polypropylene cages, in controlled environment (temperature 25±2° C.; humidity 50-60%; light 300 lux at floor level with regular 12 h light-12 h dark cycle; noise level 50 decibel; ventilation 10-15 air changes per hour). They were provided with a standard laboratory diet (Dayal Industry, Barabanki, Lucknow) unless stated otherwise and has free access to water. The mice having fasting blood glucose values varying between 65-80 mg/dl were finally included in this study. Animals were divided into groups consisting of six animals in each. Group one was considered as control group, where as the other groups were termed as experimental groups. Rats of experimental groups were given the desired doses of the test samples i.e. 5, 10, 20, 30 and 50 mg/kg body weight. The test samples were always prepared in the vehicle 1% gum acacia. An oral glucose load of 3.0 g/kg was given to the animals of all the groups 30 min post administration of the test samples/vehicle. Blood glucose levels of the animals of all the groups were again measured at 30, 60, 90 and 120 min post glucose load. Food (not water) was removed from the cages during the experimental period. A plot was drawn between time post glucose loaded and blood glucose levels on x and y axis, respectively and the area under curve (AUC) between 0 to 120 min were calculated of each group employing Graph pad Prism. Comparing the AUC of test sample treated to that of control group determined the percent improvement in OGTT by test sample which is termed as anti-hyperglycaemic activity. Statistical comparison between the groups was done by employing Students t test.

### Results of Dose Dependent Effect on OGTT in Db/+Mice

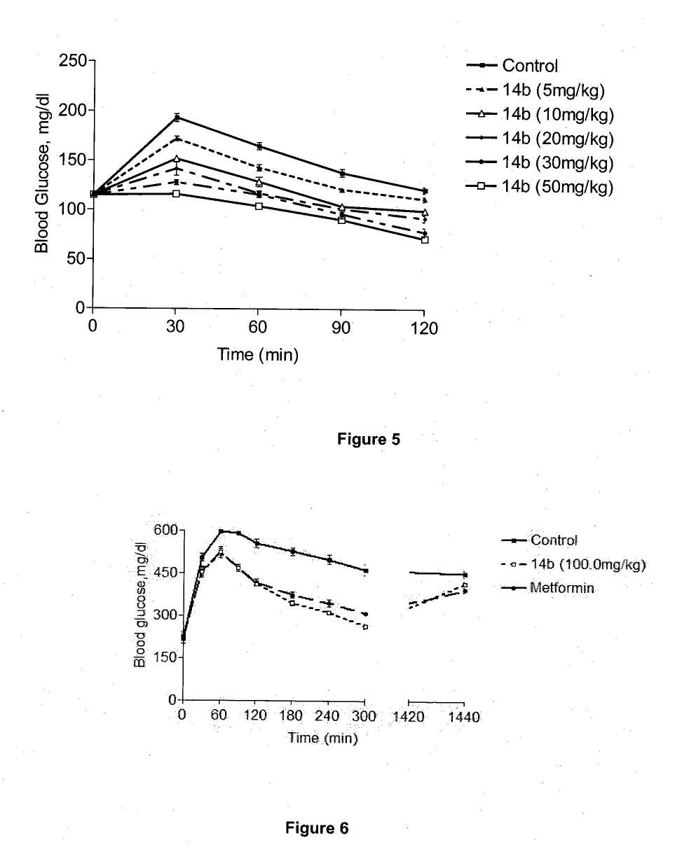

The dose dependent effect on OGTT of 14b was evaluated in db/+ mice at different dose levels like 5, 10, 20, 30 and 50 mg/kg of body weight. It is evident from the results shown in FIG. 5 that 14b improved OGTT of db+ mice to the extent of 10.5, 19.8, 24.5 (p<0.05), 28.9 (p<0.01) and 35.9% (p<0.01) over vehicle treated control at the said dose levels (Table 4 & FIG. 5).

TABLE 4
Antihyperglycaemic effect of 14b in glucose loaded db/+ mice

| | | % Antihyperglycaemic |
|---|---|---|
| Test Sample | Dose (mg/kg) | activity |
| 14b | 5 | 10.5 |
| | 10 | 19.8 |
| | 20 | 24.5* |
| | 30 | 28.9** |
| | 50 | 35.9** |

Statistical significance *p < 0.05, **p < 0.01 and ***p < 0.001

i. Sucrose Challenged Low Dosed Streptozotocin-Induced Diabetic Rats

Male albino rats (Sprague Dawley Strain) of body weight 160±20 were procured from the colony of CDRI Animal House. 4 to 5 animals were kept in one polypropylene cage and acclimatized for one week in the new environment. The animals were made diabetic by injecting streptozotocin (STZ) intraperitoneally to the overnight starved animals at a dose of 50 mg/kg prepared in 100 mM citrate buffer (pH 4.5). Fasting blood glucose level of each animal was measured after 48 hours post STZ injection by Glucostrips (Roche) using Glucometer (ACCU-CHEK II; Roche Diagnostics, USA) and animals showing blood glucose level above 280 mg/dl were considered as diabetic. The diabetic rats with fasting blood glucose values varying between 150-270 mg/dl were included in this study. Animals were divided into groups consisting of five animals in each. Group one was considered as a control group, where as the other groups were termed as experimental groups. Rats of experimental group were given the desired dose of the test samples and standard drug, (Metformin), respectively. The test samples and standard drug (Metformin) was always prepared in the vehicle 1% gum acacia. An oral sucrose load of 2.5 g/kg was given to the animals of all the groups 30 min post administration of the test samples/vehicle/standard drug. Blood glucose levels of the animals of all the groups were again measured at 30, 60, 90, 120, 180, 240, 300 min and 24 hour post sucrose load as before. Food (not water) was removed from the cages during the experimental period. A plot was drawn between time and blood glucose levels on x and y axis, respectively and the area under curve (AUC) between 0 to 5 hours were calculated of each group. The percent decline in AUC of the experiment group compared to sham treated control group termed as % antihyperglycaemic activity. Statistical analysis between the groups was done by employing Students t test.

### Antihyperglycaemic Activity of 14b and Standard Drug Metformin on Sucrose Challenged Low Dosed Streptozotocin-Induced Diabetic Rat Model

Table 5 & FIG. 6, presents the blood glucose profile post sucrose load and antihyperglycaemic effect of 14b and standard drug Metformin in sucrose challenged low dosed STZ-induced diabetic rats. It is evident from the results that there was an overall decline in the blood glucose levels of sucrose challenged low dosed STZ-induced diabetic rats by 28.9% (p<0.001) and 23.7% (p<0.01), respectively by 14b and metformin during 0-300 min and 25.2% (p<0.001) and 23.1% (p<0.01) respectively during 0-1440 min, post sucrose load over vehicle treated control group.

TABLE 5
Antihyperglycaemic effect of 14b and standard drug Metformin on Sucrose
challenged low dosed STZ-induced diabetic rats.

| Treatment | Blood glucose level (mg/dl) min post sucrose load | | | | | |
|---|---|---|---|---|---|---|
| Group | 0 | 30 | 60 | 90 | 120 | 180 |
| Control | 222.4 ± 13.6 | 505.6 ± 14.1 | 597.8 ± 2.2 | 591.4 ± 4.8 | 555.8 ± 15.2 | 527.0 ± 12.6 |
| 14b | 222.0 ± 7.9 | 452.6 ± 18.2 | 524.8 ± 11.2 | 468.2 ± 12.6 | 412.6 ± 5.8 | 345.2 ± 9.2 |
| (100 mg/kg) | | | | | | |
| Metformin | 222.2 ± 19.9 | 457.4 ± 17.2 | 524.2 ± 18.9 | 468.2 ± 11.3 | 416.0 ± 12.2 | 373.6 ± 11.4 |
| (100 mg/kg) | | | | | | |

| | | | | % Antihyper- | |
|---|---|---|---|---|---|
| | Blood glucose level (mg/dl) | | | glycemic | |
| Treatment | min post sucrose load | | | activity | |
| Group | 240 | 300 | 1440 | 0-300 min | 0-1440 min |
| Control | 499.0 ± 17.0 | 463.6 ± 18.6 | 450.2 ± 11.6 | | |
| 14b | 311.6 ± 3.7 | 264.8 ± 8.7 | 414.8 ± 8.3 | 28.9%** | 25.2%*** |
| (100 mg/kg) | | | | | |
| Metformin | 345.0 ± 10.6 | 310.8 ± 2.6 | 392.8 ± 9.9 | 23.7%** | 23.1%** |
| (100 mg/kg) | | | | | |

Blood glucose values are Mean ± SE of 5 animals per group,
Significance: *p < 0.05, **p < 0.01 and ***p < 0.001 and ns: not significant.

TABLE 2
Antihyperglycaemic activity of test compounds on sucrose loaded rat
(SLM) and sucrose-challenged streptozotocin-induced diabetic rat (STZ-S) models.

| | | % Antihyperglycemic | | |
|---|---|---|---|---|
| | | activity | | |
| Test | | | STZ-S | |
| compounds | Structure | SLM | 0-5 hr | 0-24 hr |
| 10. | 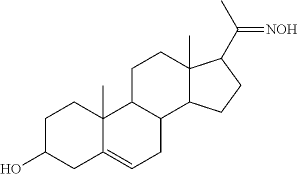 | 13.9 | ND | ND |
| 16a | 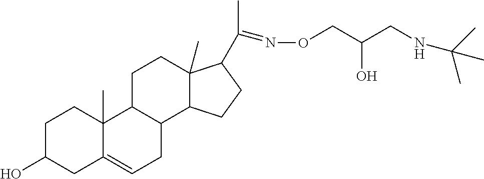 [SMILES: C/C(=N\OCC(O)CNC(C)(C)C)C1CCC2C3CC=C4CC(O)CCC4(C)C3CCC12C] | 37.7 | ND | ND |
| 16b | 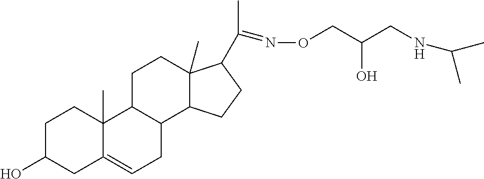 [SMILES: C/C(=N\OCC(O)CNC(C)C)C1CCC2C3CC=C4CC(O)CCC4(C)C3CCC12C] | 13.6 | ND | ND |
| 16c | 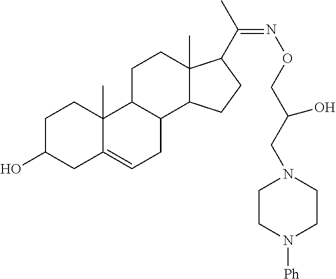 [SMILES: C/C(=N/OCC(O)CN1CCN(c2ccccc2)CC1)C1CCC2C3CC=C4CC(O)CCC4(C)C3CCC12C] | 17.8 | ND | ND |
| 16d | 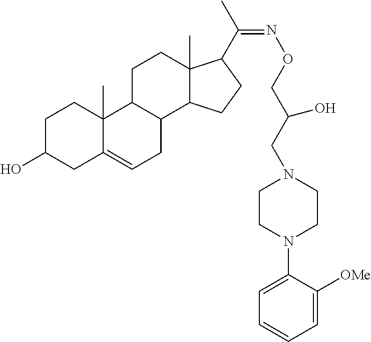 [SMILES: COc1ccccc1N1CCN(CC(O)CO/N=C(/C)C2CCC3C4CC=C5CC(O)CCC5(C)C4CCC23C)CC1] | 13.9 | ND | ND |
| 16e | 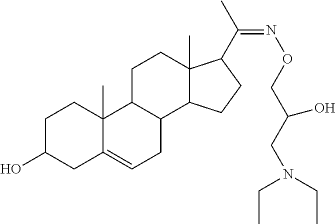 [SMILES: CCN(CC)CC(O)CO/N=C(/C)C1CCC2C3CC=C4CC(O)CCC4(C)C3CCC12C] | 17.0 | ND | ND |
| 16f | 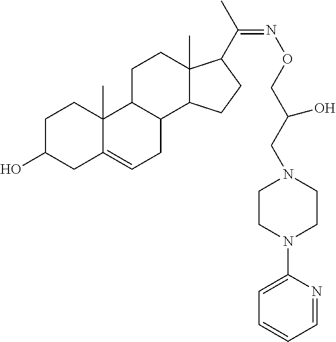 [SMILES: C/C(=N/OCC(O)CN1CCN(c2ccccn2)CC1)C1CCC2C3CC=C4CC(O)CCC4(C)C3CCC12C] | 19.6 | ND | ND |
| 14a | 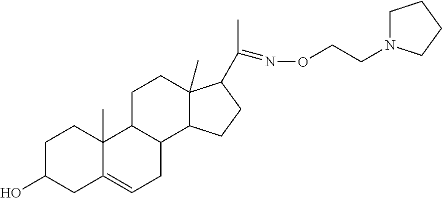 [SMILES: C/C(=N\OCCN1CCCC1)C1CCC2C3CC=C4CC(O)CCC4(C)C3CCC12C] | 42.4 | 9.96 | 3.07 |
| 14b | 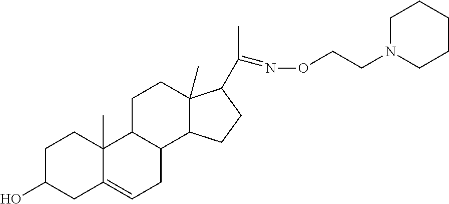 [SMILES: C/C(=N\OCCN1CCCCC1)C1CCC2C3CC=C4CC(O)CCC4(C)C3CCC12C] | 24.4 | 28.9 | 25.2 |
| 11a | 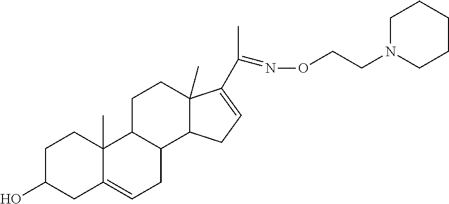 [SMILES: C/C(=N\OCCN1CCCCC1)C1=CCC2C3CC=C4CC(O)CCC4(C)C3CCC12C] | 27.15 | ND | ND |
| 11b | 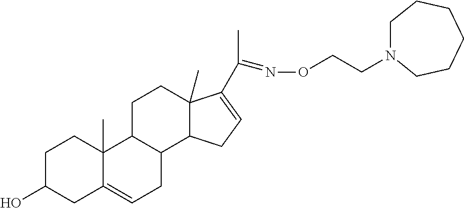 [SMILES: C/C(=N\OCCN1CCCCCC1)C1=CCC2C3CC=C4CC(O)CCC4(C)C3CCC12C] | 16.5 | ND | ND |
| 11c | 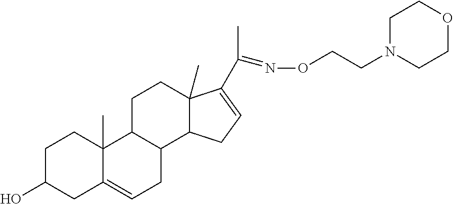 [SMILES: C/C(=N\OCCN1CCOCC1)C1=CCC2C3CC=C4CC(O)CCC4(C)C3CCC12C] | 10.2 | ND | ND |
| 11d | 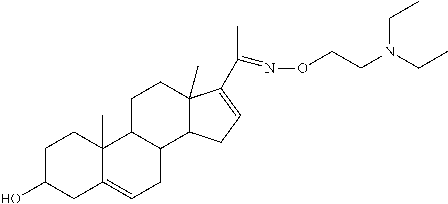 [SMILES: CCN(CC)CCO/N=C(\C)C1=CCC2C3CC=C4CC(O)CCC4(C)C3CCC12C] | 13.1 | ND | ND |
| Standard | Metformin | 21.1 | 25.8 | 33.2 |

iv. Streptozotocin-Induced Diabetic Rat Model (STZ)

Male albino rats (Sprague Dawley Strain) of body weight 160±20 g were procured from the colony of CDRI Animal House. 4 to 5 animals were kept in one polypropylene cage and acclimatized for one week in the new environment. The animals were made diabetic by injecting streptozotocin (STZ) intraperitoneally to the overnight starved animals at a dose of 60 mg/kg prepared in 100 mM citrate buffer (pH 4.5). Fasting blood glucose level of each animal was measured after 48 hours post STZ injection by Glucostrips (Roche) using Glucometer (ACCU-CHEK II; Roche Diagnostics, USA) and animals showing blood glucose level above 280 mg/dl were considered as diabetic. The diabetic rats with fasting blood glucose values varying between 280-450 mg/dl were included in this study. Animals were divided into groups consisting of five animals in each. Group one was considered as a control group, where as the other groups were termed as experimental groups. Rats of experimental group were usually given 100 mg/kg body weight of the test samples unless stated otherwise and standard drug i.e. metformin, respectively. The test samples and standard drug i.e. metformin was always prepared in the vehicle 1% gum acacia. 30 min post administration of the test samples/vehicle/standard drug, blood glucose levels of the animals of all the groups were measured at 30, 60, 90, 120, 180, 240, 300 and 1440 min. Food (not water) was removed from the cages during the experimental period. A plot was drawn between time and blood glucose levels on x and y axis, respectively and the area under curve (AUC) between 0 to 300 min were calculated of each group. The percent decline in AUC of the experiment group compared to sham treated control group termed as % antihyperglycaemic activity. Statistical comparison between the groups was done by employing Student's ‘t’ test.

### Antihyperglycaemic Activity of 14b and Standard Drug Metformin on Low Dosed Streptozotocin-Induced Diabetic Rats

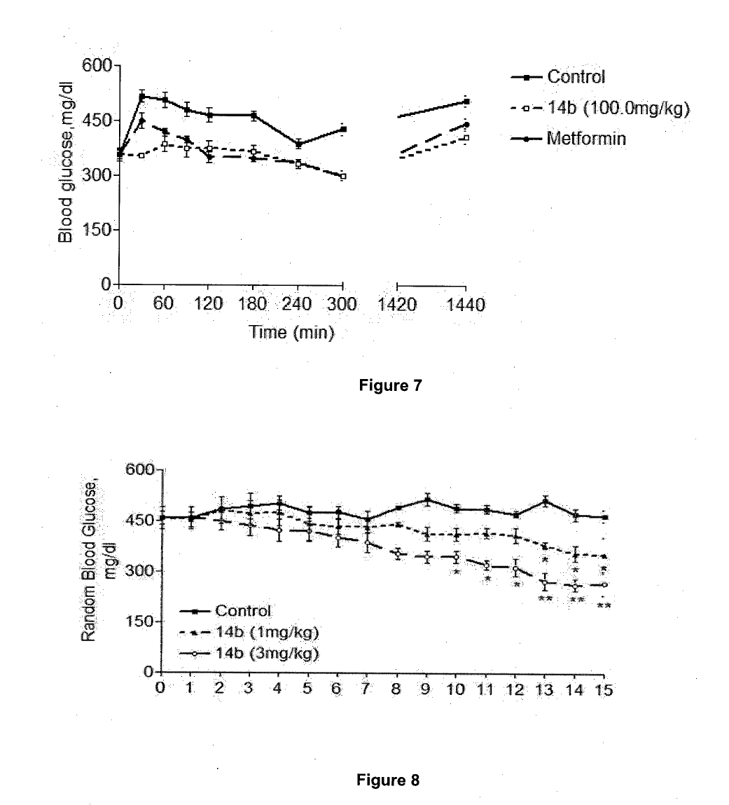

Table 6 & FIG. 7 presents blood glucose lowering effect of 14b and standard drug Metformin in low dose STZ-induced diabetic rats. The STZ-induced diabetic rats treated with 14b and Metformin, respectively at 100 mg/kg of body weight significantly declined their blood glucose level by 21.1% (p<0.01) and 19.0% (p<0.01), respectively, at 0-300 min and 22.1% (p<0.01) and 20.5%(p<0.01) at 0-1440 min compared to that of vehicle treated control group.

TABLE 6
Antihyperglycaemic effect of 14b and standard drug metformin on low dosed
STZ-induced diabetic rats

| Treatment | Blood glucose level (mg/dl) min post sucrose load | | | | | |
|---|---|---|---|---|---|---|
| Group | 0 | 30 | 60 | 90 | 120 | 180 |
| Control | 358.8 ± 9.2 | 516.8 ± 17.0 | 506.0 ± 21.5 | 480.8 ± 20.1 | 465.6 ± 18.9 | 464.4 ± 13.8 |
| 14b | 357.4 ± 17.9 | 353.8 ± 6.3 | 387.0 ± 20.8 | 374.4 ± 23.1 | 375.0 ± 20.2 | 366.6 ± 16.7 |
| (100 mg/kg) | | | | | | |
| Metformin | 358.4 ± 14.5 | 450.0 ± 21.8 | 419.0 ± 10.5 | 399.0 ± 7.7 | 351.0 ± 16.0 | 349.6 ± 10.1 |
| (100 mg/kg) | | | | | | |

| | | | | % Antihyper- | |
|---|---|---|---|---|---|
| | Blood glucose level (mg/dl) | | | glycemic | |
| Treatment | min post sucrose load | | | activity | |
| Group | 240 | 300 | 1440 | 0-300 Min | 0-1440 min |
| Control | 388.4 ± 12.6 | 428.4 ± 16.5 | 508.0 ± 15.0 | | |
| 14b | 333.0 ± 11.7 | 301.4 ± 12.1 | 408.0 ± 17.1 | 21.1%** | 22.1%*** |
| (100 mg/kg) | | | | | |
| Metformin | 337.0 ± 7.7 | 299.8 ± 5.0 | 445.6 ± 13.9 | 19.0%** | 20.5%** |
| (100 mg/kg) | | | | | |

Blood glucose values are Mean ± SE of 5 animals per group,
Significance: *p < 0.05, **p < 0.01 and ***p < 0.001 and ns: not significant

v. db/db Mice

Male C57BL/KsJ-db/db mice (8 to 12 weeks old, 35-45 g body weight) were procured from National Animal Laboratory Centre (NALC) of Central Drug Research Institute (CDRI), Lucknow, India. Animals were housed in polypropylene cages, in controlled environment (temperature 25±2° C.; humidity 50-60%; light 300 lux at floor level with regular 12 h light-12 h dark cycle; noise level 50 decibel; ventilation 10-15 air changes per hour). They were provided with a standard laboratory diet (Dayal Industry, Barabanki, Lucknow) unless stated otherwise and had free access to water. The animals were allocated into groups of 5 animals in each. Prior to start of the test sample feeding, a vehicle training period was followed from day −3 to day 0 during which all the animals were given vehicle (1% gum acacia) at a dose volume of 10 ml/kg body weight. At day 0 the animals having blood glucose level between 180 to 300 mg/di were selected and divided into three groups containing 5 animals in each. One group was considered as control group while the other group one was treatment group. The treatment groups were given suspensions of 14b at 1.0 and 3.0 mg/kg body weight, dose, respectively. The control group was given an equal amount of vehicle. All the animals had free access to fresh water and normal diet. Random blood glucose of each mouse was checked daily at 11.00. On day 10^{th} and day 14^{th} oral glucose tolerance (OGTT) test was performed to study the effect of compound on glucose tolerance. Blood has been withdrawn from the retro-orbital plexus of mice eye for the estimation of lipid profile and insulin.

### Effect on Hyperglycaemia

In type 2 diabetes, elevated blood glucose level due to defect in peripheral glucose disposal results in hyperglycaemia. To observe the improvement on hyperglycaemia 14b was orally gavaged for 15 consecutive days and after dosing blood glucose of control and treated group were measured.

FIG. 8, depicts the blood glucose levels of vehicle treated sham control and 14b treated at the dose 1.0 and 3.0 mg/kg, respectively on C57BL/KsJ-db/db mice. As evident from the results shown in FIG. 8, the test sample 14b treated groups showed decline in their blood glucose levels compared to vehicle treated group. The blood glucose lowering effect was more in group treated with 3.0 mg/kg 14 b compared group treated with 1.0 mg/kg of 14b.

Table 7 and 8 represent the effect of 14b on oral glucose tolerance in db/db mice after 10 days and 15 days, respectively, at 1.0 and 3.0 mg/kg body weight doses. The overnight fasted db/db mice were subjected to an oral glucose tolerance test post 3.0 g oral glucose load. The fasting base line blood glucose values at 0 min were found lowered in all the treated groups compared to vehicle treated control group at the corresponding time because of antihyperglycaemic effect of 14b as nearly 21.2 (p<0.05), and 29.1% (p<0.01) decline in fasting blood glucose level was observed on day 10^{th} and decline of around 25.5 (p<0.01), and 35.1% (p<0.01) was observed on day 15^{th} at dose 1.0 and 3.0 mg/kg body weight doses respectively. The treatment of 14b showed significant improvement on oral glucose tolerance by 10.3 (p<0.05) and 15.1% (p<0.01) on day 10^{th} and an improvement of 16.9 (p<0.01) and 24.5% (p<0.01) on day 15^{th} at dose 1.0 and 3.0 mg/kg body weight doses, respectively.

TABLE 7
Effect of 14b on oral glucose tolerance test (OGTT) of
db/db mice on day 10^{th}

| | Blood glucose profile (mg/dl) | | | | | % |
|---|---|---|---|---|---|---|
| Treatment | min post glucose load | | | | | Antihyperglycaemic |
| Group | 0 | 30 | 60 | 90 | 120 | activity |
| Control | 268.6 ± 21.9 | 600.0 ± 0.0 | 594.0 ± 6.0 | 574.4 ± 13.8 | 551.4 ± 19.5 | |
| 14b | 211.6 ± 11.8 | 559.8 ± 24.8 | 568.0 ± 12.9 | 502.8 ± 23.6 | 437.8 ± 14.1 | 10.3* |
| (1.0 mg/kg) | | | | | | |
| 14b | 190.2 ± 12.9 | 542.2 ± 24.1 | 523.2 ± 22.7 | 482.0 ± 20.5 | 414.0 ± 9.4 | 15.1** |
| (3.0 mg/kg) | | | | | | |

Values are Mean ± SE of 5 mice;
Statistical significance, *<0.05, **<0.01

TABLE 8
Effect of 14b on oral glucose tolerance test (OGTT)
of db/db mice on day 15^{th}

| | Blood glucose profile (mg/dl) | | | | | % |
|---|---|---|---|---|---|---|
| Treatment | min post glucose load | | | | | Antihyperglycaemic |
| Group | 0 | 30 | 60 | 90 | 120 | activity |
| Control | 246.6 ± 10.2 | 600.0 ± 0.0 | 594.0 ± 6.0 | 584.4 ± 12.9 | 561.4 ± 14.9 | |
| 14b | 183.6 ± 7.86 | 499.2 ± 36.1 | 560.6 ± 14.6 | 468.8 ± 19.5 | 383.8 ± 8.46 | 16.9** |
| treated | | | | | | |
| (1.0 mg/kg) | | | | | | |
| 14b | 160.2 ± 6.88 | 458.8 ± 16.6 | 513.2 ± 13.1 | 420.0 ± 13.2 | 348.0 ± 13.2 | 24.5** |
| treated | | | | | | |
| (3.0 mg/kg) | | | | | | |

Values are Mean ± SE of 5 mice;
Statistical significance, *<0.05, **<0.01

### Effect on Serum Lipid Profile and Insulin Level

Insulin resistance to insulin responsive organs in type 2 diabetes, is one causative factor of altered serum lipid profile and hyperinsulinemia. Improvement in diabetic conditions may also associate with the improvement in serum lipid profile.

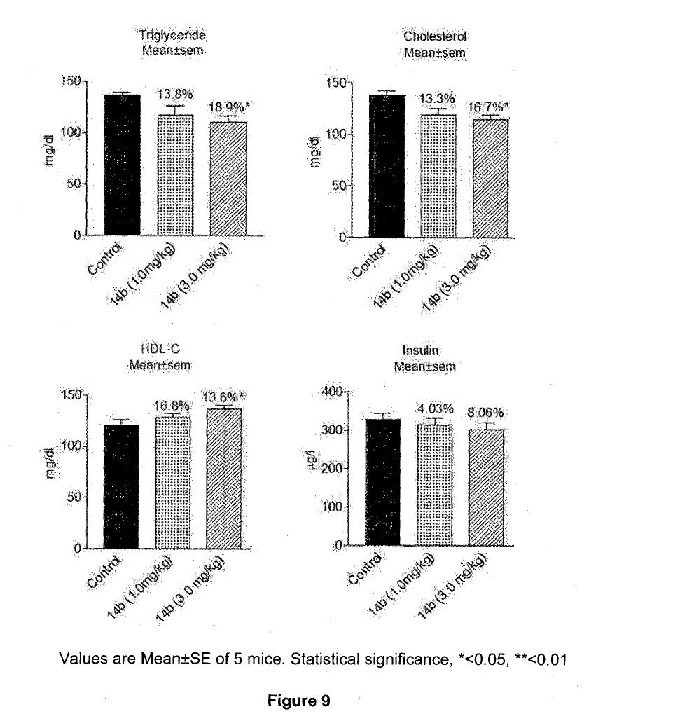

FIG. 9, represents the effect of 14b on serum lipid profiles and insulin level in db/db mice at 1.0 and 3.0 mg/kg dose level. The oral dose for 15 consecutive days was efficient in lowering the serum triglycerides level (TG) by 13.8 and 18.9% (p<0.05), and total cholesterol (T-Chol) level by 13.3 and 16.7% (p<0.05), respectively. The level of HDL-cholesterol was found enhanced by 6.18 and 13.6% (p<0.05), respectively at these dose level. 14b treatment also resulted in mild decrease in plasma insulin levels by 4.03 and 8.06% compared to vehicle treated control group.

vi. Antidyslipidemic Activity Evaluation on High Fructose High Fat Fed Syrian Golden Hamsters

Male Syrian golden hamsters weighing 100-120 g were divided into groups each containing six animals. Dyslipidemia was produced by feeding the animals with high fructose high fat diet (60% fructose, 13% fat) for 30 to 40 days. Dyslipidemic hamsters were divided into groups based on their Serum lipid profile. The dyslipidemic animals had free access to HFD and water throughout the experimental period. The fine suspension of 14b was fed orally at a dose of 30 mg/kg for 7 days to the animals. Control animals were given drug vehicle only, served as sham treated control. Body weight and diet intake of each animal group were recorded daily to check the effect of the test samples on food intake and body weight of the animals. At the end of the experiment period i.e. on day 8^{th}, the blood of each animal was withdrawn from retro-orbital plexus. After keeping the tubes for 15 min. in cold, Serum was separated. Biochemical analysis of Serum was performed on the same day for triglycerides (TG), total cholesterol (TC) and HDL-cholesterol and LDL-cholesterol contents using Cobas Integra 400 plus analyzer kits. All statistical analysis will be performed with Graph Pad Prism Software. One way ANOVA, all pair wise multiple comparison procedure (Tukey test). Data will be reported as mean±SEM.

### Antidyslipidaemic Activity Evaluation in High Fructose High Fat Fed Syrian Golden Hamsters

High fructose high fat fed Syrian golden hamster model is one of the best model for the evaluation of antidyslipidemic agents. To verify the presence of antidyslipidemic properties in 14b, it was orally gavaged at 30 mg/kg dose level.

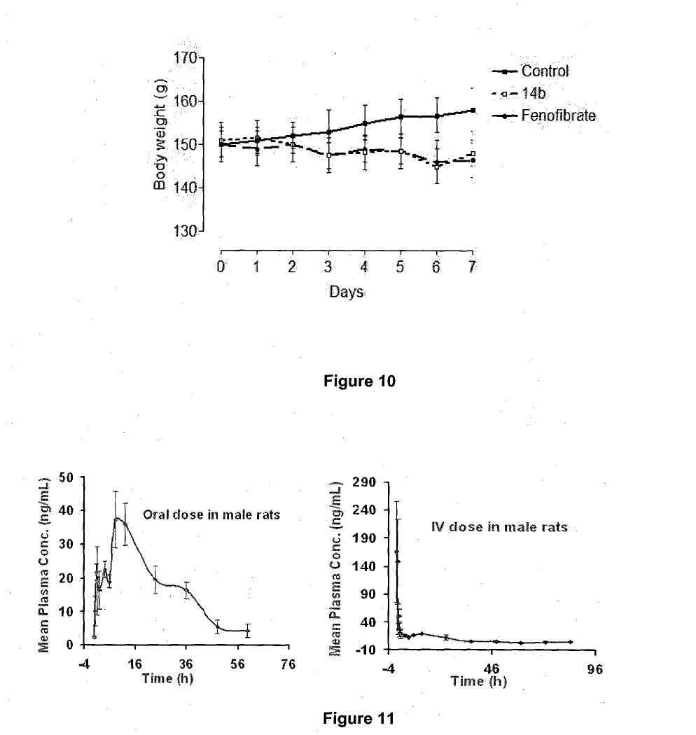

Table 9 represent the antidyslipidemic effect of 14b in high fructose high fat fed Syrian golden hamsters at 30 mg dose level. This oral dose of 14b was found efficient in lowering the serum triglycerides level (TG) by 32.8% (p<0.05), total cholesterol (T-Chol) level by 23.1% (p<0.05), and LDL-cholesterol (LDL-C) level by 32.3% (p<0.05), serum glycerol level by 25.7%, non-esterified fatty acid (NEFA) by 12.3% where as the standard drug fenofibrate showed an improvement of 28.9% (p<0.05), 16.9%, 30.1% (p<0.05), 22.4% (p<0.05) and 16.9% on serum triglycerides (TG), total cholesterol T-Chol) and LDL-cholesterol (LDL-C), glycerol and non-esterified fatty acids (NEFA) at the same dose level, respectively. The level of serum HDL-cholesterol and lipoprotein lipase was also found enhanced by 17.8 and 19.4% in 14b treated group and an increase of 9.8% and 21.1% (p<0.05) by standard drug fenofibrate, at this dose level. It was also found that 14b effectively inhibited the rise in body weight of high fructose high fat fed Syrian golden hamsters compared to the control group. The results are shown in FIG. 10.

TABLE 9
Antidyslipidemic activity evaluation of 14b in high fructose high fat fed male Syrian
golden hamsters

| | Chol | TG | HDL-C | LDL-C | Glycerol | NEFA | Lipoprotein |
|---|---|---|---|---|---|---|---|
| Groups | (mg/dl) | (mg/dl) | (mg/dl) | (mg/dl) | (μmol/l) | (mmol/l) | lipase (U/L) |
| HFD Control | 334.6 ± 37.8 | 747.1 ± 64.9 | 142.8 ± 6.80 | 265.5 ± 26.6 | 1353.0 ± 75.5 | 6.50 ± 0.40 | 30.4 ± 3.70 |
| (1% gum | | | | | | | |
| acacia) | | | | | | | |
| HFD + 14b | 257.2 ± 22.4 | 501.8 ± 45.7 | 168.2 ± 8.90 | 179.6 ± 13.2 | 1004.9 ± 40.2 | 5.70 ± 0.42 | 36.3 ± 3.90 |
| (30 mg/kg p.o.) | | | | | | | |
| | (−23.1)* | (−32.8)* | (+17.8) | (−32.3)* | (−25.7) | (−12.3) | (+19.4) |
| HFD + | 277.8 ± 22.3 | 529.8 ± 30.7 | 156.8 ± 12.8 | 185.1 ± 11.2 | 1050.2 ± 45.5 | 5.40 ± 0.30 | 36.8 ± 3.60 |
| Fenofibrate | | | | | | | |
| (30 mg/kg p.o.) | (−16.9) | (−28.9)* | (+9.80) | (−30.1)** | (−22.4)* | (−16.9) | (+21.1)* |

Data are mean ± SE values of six hamsters per group;
***p < 0.001, **p < 0.01, *p < 0.05 as compared to HFD control.

### Method Development and Pharmacokinetic Study of Compound 14b

In above studies compound 14b was found to one the best compound as antidiabetic and antidyslipidemic and was therefore selected for further study. In order to study its bioavailability, the method of its quantitative determination in plasma matrices in rat was developed and validated. The method was found to be sensitive, selective, accurate and precise over the range 1.56-200 ng/ml. The method was applied in the analysis of PK samples obtained after oral and intravenous dose administration in rats.

### Pharmacokinetic Study

The compound 14b shows slow absorption and its elimination half-life is found to be >14 hrs. The MRT value of 25.29±1.99 h after an intravenous dose and 23.23±1.31 h after oral dose indicates that 14b is retained in the system for longer periods of time due to slow elimination from the body. As a result it exhibits high volume of distribution (738.69±87.13 L) which suggests good distribution outside vascular compartment. After oral dosing with 14b, it appears that the absorption is slow as plasma concentrations peaked at ≧10 hr post-dose (table-10 and 11). The systemic bioavailability of the compound is found to be 33.61% after oral administration. The large clearance of the compound indicates a high extraction ratio across the eliminating organs (FIG. 11).

### Pharmacokinetic Parameters for 14b

TABLE 10
Oral Route:

| Parameters | (Compound 14b) |
|---|---|
| Cₘₐₓ (ng/ml) | 40.35 ± 7.03 |
| T_{1/2} (h) | 14.27 ± 0.95 |
| AUC_{0-inf} (ng · h/ml) | 1209.48 ± 166.47 |
| Vz/F (L) | 738.69 ± 87.13 |
| Cl/F (L/h/Kg) | 36.19 ± 4.55 |
| MRT (h) | 23.23 ± 1.31 |
| % Bioavailability | 33.61 |

TABLE 11
Intravenous route

| Parameters | (Compound 14b) |
|---|---|
| C₀ (ng/ml) | 166.28 ± 90.25 |
| T_{1/2} (h) | 15.42 ± 1.25 |
| AUC₀₋ₗₐₛₜ (ng · h/ml) | 825.58 ± 88.71 |
| Vz (L) | 267.52 ± 11.33 |
| Cl (L/h/Kg) | 12.34 ± 1.37 |
| MRT (h) | 25.29 ± 1.99 |

### Stability Studies

The plasma of the rat treated with compound 14b was then processed for the stability study. The compound is found to be stable during freeze-thaw cycle, on the bench top, dry residue and long term conditions after extraction from plasma. It exhibits low stability in simulated gastric fluid (SGF) while good stability in simulated intestinal fluid (SIF).

We claim:

1. A compound of formula 3 or a pharmaceutically acceptable salt thereof: 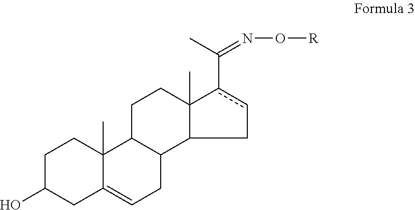
wherein R is selected from the group consisting of hydrogen (H), n-butyl, benzyl or 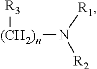
where n is 2 or 3,
R1 and R2 are independently selected from the group consisting of H and an alkyl group, or R1 and R2 together form a cyclic system wherein the cyclic system is selected from the group consisting of 4-phenyl-piperazine-1-yl, 4-(2-methoxy phenyl)-piperazinyl, pyrrolidinyl, piperidinyl, azepanyl and morpholine;
R3 is H or OH,
wherein the alkyl group is selected from the group consisting of ethyl, isopropylamine, diisopropyl and t-butyl amine.

2. The compound as claimed in claim 1, selected from the group consisting of:
3β-Hydroxy-pregna-5,16-dien-20-one-O-n-butyl-oxime (10a),
3β-Hydroxy-pregna-5,16-dien-20-one-O-benzyl-oxime (10b),
3β-Hydroxypregna-5,16-dien-20-one-O-(2-piperidinyl-ethyl)-oxime (11a),
3β-Hydroxypregna-5,16-dien-20-one-O-(2-azepan-1-yl-ethyl)-oxime (11b),
3β-Hydroxypregna-5,16-dien-20-one-O-(2-morpholin-4-yl-ethyl)-oxime (11c),
3β-Hydroxypregna-5,16-dien-20-one-O-(2-diethylamino-ethyl)-oxime (11d),
3β-Hydroxy-pregna-5,16-dien-20-one-O-[3-{2-hydroxy-3-(4-(2-methoxy-phenyl-piperazinyl)-propyl)}]-oxime (13a),
3β-Hydroxy-pregna-5,16-dien-20-one-O[3-(2-hydroxy-3-(4-phenyl-piperazinyl)-propyl)]-oxime (13b),
3β-Hydroxy-pregna-5-en-20-one-O-(2-pyrrolidin-1-yl-ethyl)-oxime (14a),
3β-Hydroxy-pregna-5-en-20-one-O-(2-piperidin-1-yl-ethyl)-oxime (14b),
3β-Hydroxy-pregna-5-en-20-one-O[2-hydroxy-3-iso-propylamino-propyl)-oxime (16a),
3β-Hydroxy-pregna-5-en-20-one-O-[2-hydroxy-3-tert.-butylamino-propyl]-oxime (16b),
3β-Hydroxy-pregna-5-en-20-one-O-[2-hydroxy-3-(4-phenyl-piperazin-1-yl)-propyl]-oxime (16c),
3β-Hydroxy-pregna-5-en-20-one-O-{2-hydroxy-3-[4-(2-methoxyphenyl)-piperzin-1-yl]-propyl}-oxime (16d),
3β-Hydroxy-pregna-5-en-20-one-O[2-hydroxy-3-(2-pyridyl-piperazin-1-yl)-propyl]-oxime (16f),
3β-Hydroxy-pregna-5-en-2-one-O-[2-hydroxy-3-pipridinyl-propyl)-oxime (16g),
3β-Hydroxy-pregna-5-en-20-one-O-[2-hydroxy-3-(4-methyl-piperazin-1-yl)-propyl]-oxime (16h),
3β-Hydroxy-pregna-5-en-20-one-O-[2-hydroxy-3-diisopropylamino-propyl)-oxime (16i), and
3β-Hydroxy-pregna-5-en-20-one-O-[2-hydroxy-3-diethylamino-propyl)-oxime (16j).

3. The compound as claimed in claim 1, wherein the compound is useful for the treatment of diabetes and dyslipidemia.

4. A process for preparation of the compound of formula 3 as claimed in claim comprising the steps of:
I. reacting a compound of formula A 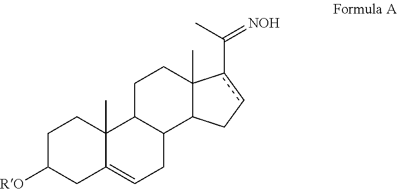
wherein R′ is selected from COCH3 or H,
with an alkyl halide, a benzyl halide, a substituted aminoethylhalide or an epoxy propylhalide in presence of a base, in a solvent, to obtain a reaction mixture;
ii. evaporating the reaction mixture obtained from step (i), under vacuum, followed by extraction with a water immiscible solvent and purification to obtain corresponding compounds of formula 10(a-b), 11(a-d), 12, 14(a-b) and 15, 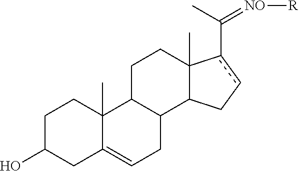
wherein, R is selected from the group consisting of hydrogen (H), n-butyl, benzyl, substituted aminoethyl and epoxy propyl, or cyclic aminoethyl,
10(a-b): R: butyl or benzyl, 11(a-d): R=piperidinyl-ethyl or azepan-1-yl-ethyl or morpholin-4-yl-ethyl or diethylamino-ethyl, 12: and 15: R=epoxypropyl,
14(a-b): R=pyrrolidin-1-yl-ethyl or piperidin-1-yl-ethyl;
iii. further comprising the step of reacting compound 12 or 15 obtained from step (ii) with an amine in methanol, under reflex, followed by purification, to obtain compounds 13(a-b) and 16(a-j), 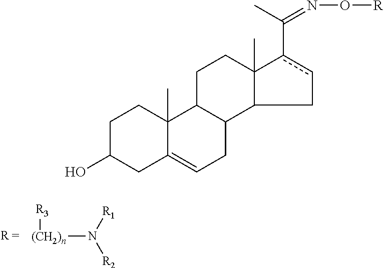
where n is 2 or 3,
R1 and R2 are independently selected from the group consisting of hydrogen (H) and an alkyl group, or R1 and R2 together form a cyclic system wherein the cyclic system is selected from the group consisting of 4-phenyl-piperazine-1-yl, 4-(2-methoxy phenyl)-piperazinyl, pyrrolidinyl, piperidinyl, azepanyl and morpholine,
R3 is H or OH,
wherein the alkyl group is selected form the group consisting of ethyl, isopropylamine, diisopropyl and t-butyl amine.

5. The process as claimed in claim 4, wherein the water immiscible solvent of step (ii) is selected form the group consisting of chloroform, dichloromethane, ether and ethyl acetate.

6. The process as claimed in claim 4, wherein the reaction between the compound of formula A and the alkyl halide, benzyl halide, substituted aminoethylhalide or epoxy propylhalide in step (i) is carried out at a temperature ranging from 30 to 80 degree C. for a period ranging from 10 to 20 hrs.

7. The process as claimed in claim 4, wherein the solvent of step (i) is selected from the group consisting of DMF and N-methylpyrrolidone.

8. The process as claimed in claim 4, wherein the base of step (i) is selected from the group consisting of sodium hydride and potassium hydride.

9. A pharmaceutical composition comprising a pharmaceutically effective amount of a compound of formula 3 or a pharmaceutically acceptable salt thereof as claimed in claim 1, optionally along with pharmaceutically acceptable additives.

10. A composition as claimed in claim 9, wherein said composition is useful for the treatment of diabetes and dyslipidemia.

11. A method for treating type II diabetes, comprising the step of administering to a subject in need thereof a pharmaceutically effective amount of a compound of formula 3 or pharmaceutically acceptable salt thereof, 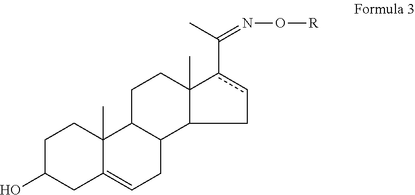
wherein R is selected from the group consisting of hydrogen (H), n-butyl, benzyl or 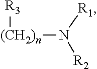
where n is 2 or 3,
R1 and R2 are independently selected from the group consisting of H and an alkyl group, or R1 and R2 together form a cyclic system wherein the cyclic system is selected from the group consisting of 4-phenyl-piperazine-1-yl, 4-(2-methoxy phenyl)-piperazinyl, pyrrolidinyl, azepanyl, piperidinyl and morpholine;
R3 is H or OH,
wherein the alkyl group is selected form the group consisting of ethyl, isopropylamine, diisopropyl and t-butyl amine,
optionally along with other antidiabetic and antidyslipidemic agents.
